SEQUENCE LISTING (46 sequences)

SEQ ID NO: 1 - DNA, 144 bp, Triticum aestivum
  source 1..144: mol_type = genomic DNA
  misc_feature 1..144: note = EN1390
tagacacgttgtgagctgccttgacttgggtaggcagatccagacctgaaacgtcaggctcatggataaggatcccggccatctgtcactgacgtgtcgtcagtttgcccctagccttgtgggattgccatctccatctgccta

SEQ ID NO: 2 - DNA, 144 bp, synthetic construct
  source 1..144: mol_type = other DNA
  misc_feature 1..144: note = EN1390opt
tagacacgttgtgagctgccttgacttgggtaggcagatccagacctgaaacgtcaggctcatggataatgatcccggccacctgggcatgacgtggcgtcagtttgcccctagccttgtgggattgccatctccatctgccta

SEQ ID NO: 3 - DNA, 17 bp, Triticum aestivum
  source 1..17: mol_type = genomic DNA
  misc_feature 1..17: note = EN1390(nt81-97wt)
atctgtcactgacgtgt

SEQ ID NO: 4 - DNA, 17 bp, synthetic construct
  source 1..17: mol_type = other DNA
  misc_feature 1..17: note = EN1390(nt81-97mut)
acctgggcatgacgtgg

SEQ ID NO: 5 - DNA, 41 bp, Triticum aestivum
  source 1..41: mol_type = genomic DNA
  misc_feature 1..41: note = EN1390(nt61-101wt)
catggataaggatcccggccatctgtcactgacgtgtcgtc

SEQ ID NO: 6 - DNA, 41 bp, synthetic construct
  source 1..41: mol_type = other DNA
  misc_feature 1..41: note = EN1390(nt61-101mut)
catggataatgatcccggccacctgggcatgacgtggcgtc

SEQ ID NO: 7 - DNA, 144 bp, Triticum aestivum
  source 1..144: mol_type = genomic DNA
  misc_feature 1..144: note = EN3233
atgtatgcagccgtctcctgctgcttcactcaaggacgccacgccatccacgcatatccggtggcggcaaacgccgtccaggttcaacgaacgccgttccccccggtccaccagcgccagccgcctccggccgggcccacccgc

SEQ ID NO: 8 - DNA, 144 bp, synthetic construct
  source 1..144: mol_type = other DNA
  misc_feature 1..144: note = EN3233opt
atgtatgcagccgtctcctgctgcttcactcaaggacgccacgccatccacgcatatccggtggcggcaaacgccgtccaggttcaacgaacgtcgttccccccggtccaccagcgccagccgcctccggccgggcccacccgc

SEQ ID NO: 9 - DNA, 16 bp, Triticum aestivum
  source 1..16: mol_type = genomic DNA
  misc_feature 1..16: note = EN3233(nt79-94wt)
caggttcaacgaacgc

SEQ ID NO: 10 - DNA, 16 bp, synthetic construct
  source 1..16: mol_type = other DNA
  misc_feature 1..16: note = EN3233(nt79-94mut)
caggttcaacgaacgt

SEQ ID NO: 11 - DNA, 27 bp, Triticum aestivum
  source 1..27: mol_type = genomic DNA
  misc_feature 1..27: note = EN3233(nt74-100wt)
ccgtccaggttcaacgaacgccgttcc

SEQ ID NO: 12 - DNA, 27 bp, synthetic construct
  source 1..27: mol_type = other DNA
  misc_feature 1..27: note = EN3233(nt74-100mut)
ccgtccaggttcaacgaacgtcgttcc

SEQ ID NO: 13 - DNA, 21 bp, Triticum aestivum
  source 1..21: mol_type = genomic DNA
  misc_feature 1..21: note = EN3233(nt106-126wt)
gtccaccagcgccagccgcct

SEQ ID NO: 14 - DNA, 21 bp, synthetic construct
  source 1..21: mol_type = other DNA
  misc_feature 1..21: note = EN3233(nt106-126mut)
atcgaccaacgccagccgcct

SEQ ID NO: 15 - DNA, 144 bp, Triticum aestivum
  source 1..144: mol_type = genomic DNA
  misc_feature 1..144: note = EN5458
cagtacgatcaatgcagtgactaagtgccgtgctgtcaagtgcgtactggtaatcgggatagaaccagctcgtcatcgccagcacctagaaagagtagatagatagatgccccggtcgacacgataggataaccatcggcgcgg

SEQ ID NO: 16 - DNA, 144 bp, Triticum aestivum
  source 1..144: mol_type = genomic DNA
  misc_feature 1..144: note = EN2393
gtctccgtccggaacgtccatcgccgccgcttcctcgtcgtggccgccgcgtgcgggctggaaagggacggccagcttttccgacgcctccctgacgtgtcagcccagccccgaaccaccacacgcgtccatccccatccgctg

SEQ ID NO: 17 - DNA, 144 bp, Triticum aestivum
  source 1..144: mol_type = genomic DNA
  misc_feature 1..144: note = EN2968
ttcccccgcaaaagaaaaagtacaaggaggcatgcagcatggcgtccatgcatgctgctagatgacgtgtacccttgagcggttcagcctgttcatcctctccaaagggaaataaaaaaaaatgtccacgtgcatcgaaccgga

SEQ ID NO: 18 - DNA, 144 bp, Triticum aestivum
  source 1..144: mol_type = genomic DNA
  misc_feature 1..144: note = EN1391
ttgccatctccatctgcctaaccctcacccgatgcgtgtcctgtttcttgggggttcggtgattggtgtcaccaatttgtttcttctttgattgggacctgtacagtggaagatttactcttggaattgtctgccgggatagga

SEQ ID NO: 19 - DNA, 144 bp, Triticum aestivum
  source 1..144: mol_type = genomic DNA
  misc_feature 1..144: note = EN4730
atgcggctagatgacgtgtaccctttgagcggttcatcctgtccaacgtgaaaaaaaatgtccacgtgcatcgaaccggacaataggccagcccacactgcccatgccatcacgtttcatgcagcgggagaatattttacgggg

SEQ ID NO: 20 - DNA, 144 bp, Triticum aestivum
  source 1..144: mol_type = genomic DNA
  misc_feature 1..144: note = EN3681
gcccgtggccgccgagcgctgggcttgtttattcggagggaggcggccgagatgcgcaggtgcgacgggtggattgaccccggttcgtgtgggtacgttgaacctggacgtctttttcggtgagaaaagcacgattcgattccc

SEQ ID NO: 21 - DNA, 144 bp, Triticum aestivum
  source 1..144: mol_type = genomic DNA
  misc_feature 1..144: note = EN5128
gaattggtgagtgggtgaagggaggcaagacttgacccagagcacgcatctacgtggactcctcggtgggtccatccgtcatcacgcccacatggcgtcctcccattggccacaaccccgcccgggagcctctgattggccgga

SEQ ID NO: 22 - DNA, 144 bp, Triticum aestivum
  source 1..144: mol_type = genomic DNA
  misc_feature 1..144: note = EN3638
cgattttcttttaggaagaaaatggtatggagattccaaattctcctctcctttccttttgcgcaagaaagaaagaagatgggggggcggagagaatggccgagttgtgaaatctaaacacgaagggtcaaataatacggggta

SEQ ID NO: 23 - DNA, 144 bp, Triticum aestivum
  source 1..144: mol_type = genomic DNA
  misc_feature 1..144: note = EN2516
atgcgccatgtcgccgtcacgtaccgctcatgggtttcgtgcccggcccagtttcgcaacgctcgcttgcgacacgcactatgtgacctcagccactccatccggatatggacacccgcttttttcctcacctgccctcggaag

SEQ ID NO: 24 - DNA, 144 bp, Triticum aestivum
  source 1..144: mol_type = genomic DNA
  misc_feature 1..144: note = EN2161
tagactcggttgcttttcataaaactaactttgtattttcctctaagatgataagatgaaatgacgcctgtagagcatgacatgactcattgaattccctttacacgtaaaggatggtaattgcttacaagctactcctactag

SEQ ID NO: 25 - DNA, 144 bp, Triticum aestivum
  source 1..144: mol_type = genomic DNA
  misc_feature 1..144: note = EN2162
aaaactaactttgtattttcctctaagatgataagatgaaatgacgcctgtagagcatgacatgactcattgaattccctttacacgtaaaggatggtaattgcttacaagctactcctactagcatatatccggtcctaactc

SEQ ID NO: 26 - DNA, 144 bp, Triticum aestivum
  source 1..144: mol_type = genomic DNA
  misc_feature 1..144: note = EN5096
attgggcgtcaaataggctacagcacccaggtcgccaggtcggtcctcttaccggagtcaaacccaaccacgcgcttccctctgcgccgaaccctcctccccctttcgccagaacaccccggaagcatctggaagcccggccct

SEQ ID NO: 27 - DNA, 144 bp, Triticum aestivum
  source 1..144: mol_type = genomic DNA
  misc_feature 1..144: note = EN5216
ccgtgtgtgtgagatttatatacggcggggctggaggtggtcagatccgcaggctgcccccgtcgcgctggccggaacgtcacggggccaagctgggcgaatcgttggcgggcacgtggcggcccgggccaatcggggcgggct

SEQ ID NO: 28 - DNA, 144 bp, Triticum aestivum
  source 1..144: mol_type = genomic DNA
  misc_feature 1..144: note = EN4689
tttttcttctgcataaaaatttcgcgtggggaagctctgttcagagaccagccaggcagtgggcacgtctcctactagagtcacgggacgctctcgtcgggttgtccgtagagcgtctaggtactctgtacgaagcactgcacc

SEQ ID NO: 29 - DNA, 144 bp, Triticum aestivum
  source 1..144: mol_type = genomic DNA
  misc_feature 1..144: note = EN2470
tagagaaacttctatccttagccaaaaagcaccgtgaaggccaagttttgagaagtcaaataaactaattgaccaagaagaagaagtctaaccagcttgtgaagcaagttttcttatggactgaaaaatgggtggctatatata

SEQ ID NO: 30 - DNA, 144 bp, Triticum aestivum
  source 1..144: mol_type = genomic DNA
  misc_feature 1..144: note = EN1451
tagatcgcgagattgtgctccgatgtgcttccaaccgagtgctcttcctcgggctttagtcggtgtgtcgatcgatagtgtgatctggggtgacgtgaaggggttgtcttcttactgtttactggatcgtgttgcattgtctca

SEQ ID NO: 31 - DNA, 144 bp, Triticum aestivum
  source 1..144: mol_type = genomic DNA
  misc_feature 1..144: note = EN1380
tagatcgcgagattgtgctccgatgtgcttccaaccgagtgctcttcctcgggcttcagtcggtgtgccgatcgatagtgtgatctggggtgacgtgaaggggttgtcttcctactggttcatgaatcgtgatgcattgtctca

SEQ ID NO: 32 - DNA, 144 bp, Triticum aestivum
  source 1..144: mol_type = genomic DNA
  misc_feature 1..144: note = EN3504
ccacaccatccacaagtcgccgggcccaacggacccgtcgtcccgatcccccaacctcccgtcgtctcccttcccgcacccggtagaacgttcgggaacccctcctccgactatataaccctgccccccgcgcccgtcccctcc

SEQ ID NO: 33 - DNA, 144 bp, Triticum aestivum
  source 1..144: mol_type = genomic DNA
  misc_feature 1..144: note = EN3095
tcctggtccctgtggggtattatccatttttttctggttgattgattgaaaccttatccagtcattatcgacgtggaaagcctggagggtctgccgggtatcccgcggatcggattagattaagcgcgtccagctatgagctaa

SEQ ID NO: 34 - DNA, 144 bp, Triticum aestivum
  source 1..144: mol_type = genomic DNA
  misc_feature 1..144: note = EN4474
ggcttcggaatgcggcttgcgtgccacggaacttgttctctgtggattggatacagagcatcgtttgctttgagacgtgtcagggctatcgctttccggcggccggataaacacctcaaacgtcaacgtcatgtgcgtaaatct

SEQ ID NO: 35 - DNA, 144 bp, Triticum aestivum
  source 1..144: mol_type = genomic DNA
  misc_feature 1..144: note = EN1549
cgacgtctcgtcgccgtctaggttttatttattttccggatcgcgtcgcgatcgcgcccactgttgcttcaagaacagctaagcctgaacgagtcgtggttgttgaccgtggactgtcgctgtagcttgatctttactgcactt

SEQ ID NO: 36 - DNA, 144 bp, Triticum aestivum
  source 1..144: mol_type = genomic DNA
  misc_feature 1..144: note = EN4245
gttggggggattagcgatagggtttatatacagggacgtgaccgcgtgagggacggtggtgggtctccgtctacgacgacttggcgtcatgcctcggatgccaagtgcggatgcttccttcgcaccgaaggttggagaaccgcc

SEQ ID NO: 37 - DNA, 144 bp, Triticum aestivum
  source 1..144: mol_type = genomic DNA
  misc_feature 1..144: note = EN1267
cccagatcccaacgaccgcacgcgcgggcgcgcgcagagggcacgtttccccaccaatggcgtcgtctactacccgcggagataaacccaccaccagaaaaataaattccaaccccctgccctgccgccgaggtggggcagaga

SEQ ID NO: 38 - DNA, 144 bp, Triticum aestivum
  source 1..144: mol_type = genomic DNA
  misc_feature 1..144: note = EN3758
cgcaggaaccgaacactatggccactgtggcttgtcaaaacgagagagagagacctgcgaatcaaatgacgcaccacctaatcacgaaagtagggtgagcacgtaccgctttgactagcgtggaaaattggggcggccctcgga

SEQ ID NO: 39 - DNA, 144 bp, Triticum aestivum
  source 1..144: mol_type = genomic DNA
  misc_feature 1..144: note = EN4663
gggcgctccccggccagaataaaacgggcagtttccggcgtgaccatcggataaacgcgggcgggaaatggaactgggtgtgaacggggcgggggatgcgaatttggcagatgccgttcacatggcccgcacgcgaggcgaccg

SEQ ID NO: 40 - DNA, 144 bp, Triticum aestivum
  source 1..144: mol_type = genomic DNA
  misc_feature 1..144: note = EN2195
tccccacttttccctcgaggaaagaaaagttaaacctcgtactgtaaccgcgtcatattggcattgggccgaaaaccctacaagcccattcagacctaagatcacttgtataaagcgtcacgcgtccgtgcgccgtctccgtcg

SEQ ID NO: 41 - DNA, 107 bp, Triticum aestivum
  source 1..107: mol_type = genomic DNA
  misc_feature 1..107: note = ALMT1B
gcaggggcgaggtcgtatctggcagcggcgtgccctgaggaggtcggatccggcggaggcgcgcgctcggagaggccgtatccagcggaggcgaccggcaggggggg

SEQ ID NO: 42 - DNA, 1000 bp, Triticum aestivum
  source 1..1000: mol_type = genomic DNA
  misc_feature 1..1000: note = T6PPpromoter
gttgcggcggacaccggtcatagattctggatcccttaccgcggggcaaaaggggccgccactcacgattcacacgcatgggggaatgcggtttggaggctggttgttgtgttccaacacgtcgtctgttgcaacggtcaagttgggggaggaggctgcgcgtggtccagcttgcggcaaatcggacgactccgcagcacctgatctaactgctcgcatgagagcttacttttggcatgcatcacagccacgataaaacaaggctaacatagtcttggtccatctataatacatgttggaccatgcttctctctccccactaatcgattgctttctcctttgaccgtatttgatcttattttttcttctagtattttattttctcttgacattgggtttattggatgtgcgcggctcccgcatgtcagtgaccaacatcaaaggacactccttccgccaaagtccctctgattcttcgagtcgattttcccccttgcaacagatggctatatgtgactgatcgagaaatggccacacatttcatccaaaaatgaagaatatttgaatttccacagcctccagagcaccactttgatttgaactcgaaatatgaatatagtaaaagggtctacatataatttgaaagtattttgcaggacaaaaacaacaatgttattctcgaatcttaatctatagtcgtcaattaaattttcagaatgttaactgttcatataattgtgcaccctgcaattgtgaatgagaaaacgacacatgtccactccggttagaaaaaacgcagtagttccactagtatgggtaccgacccaacgccgctccgcctttataagtaccgacacttcgccattggcttcttcacccagggaaaaccggtctggtcgcctcttcctcgtgaactattccccactgtcactgctgggcaccactctcacgcagcgggaggcgcgtgtcgtgagcacacgtgtggttgtttgcgtgcgcc

SEQ ID NO: 43 - DNA, 144 bp, Cauliflower mosaic virus
  source 1..144: mol_type = genomic DNA
  misc_feature 1..144: note = P35S
ctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacg

SEQ ID NO: 44 - DNA, 144 bp, Triticum aestivum
  source 1..144: mol_type = genomic DNA
  misc_feature 1..144: note = ALMT1 3-prime
gcgcgtgctagggtactgtacttaattagcagcgccggcaggggcgaggtcgtatctggcagcggcgtgccctgaggaggtcggatccggcggaggcgcgcgctcggagaggccgtatccagcggaggcgaccggcaggggggg

SEQ ID NO: 45 - DNA, 144 bp, Triticum aestivum
  source 1..144: mol_type = genomic DNA
  misc_feature 1..144: note = ALMT1 5-prime
cacgccggctcgtacgtagcgccgtcgtggtgtcccctggcgactgatttgggcagcgcggtggatgggttaggaggaatgtgagcgcgccatgtgtttgtccgccagtgcctaactgccgcactgcctcaaaaggcgcgtgct

SEQ ID NO: 46 - DNA, 144 bp, Escherichia virus Lambda
  source 1..144: mol_type = genomic DNA
  misc_feature 1..144: note = lambda insulator
cagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcac

(19) United States
(12) Patent Application Publication
(10) Pub. No.: US 2026/0125694 A1
(43) Pub. Date: May 7, 2026

(54) REGULATORY NUCLEIC ACID MOLECULES FOR ENHANCING GENE EXPRESSION IN PLANTS

(71) Applicant: BASE SE, Ludwigshafen am Rhein (DE)

(72) Inventors: Frank MEULEWAETER, Gent (BE); Christophe LISERON-MONFILS, Saskatoon (CA)

(21) Appl. No.: 19/122,961

(22) PCT Filed: Oct. 10, 2023
(86) PCT No.: PCT/EP2023/078072

§ 371 Date: Apr. 21, 2025

(30) Foreign Application Priority Data
Oct. 20, 2022 (EP) 22202840.9

(51) Int. Cl. C12N 15/82

(52) U.S. Cl. CPC C12N 15/8216

(57) ABSTRACT

The current disclosure generally relates to the technical field of plant molecular biology and provides methods for the production of high expressing promoters and the production of plants with enhanced expression of nucleic acids wherein combinations of at least two nucleic acid expression enhancing nucleic acids (NEENAs) are functionally linked to said promoters and/or introduced into plants.

### FIELD OF THE INVENTION

The present invention generally relates to the technical field of plant molecular biology and provides methods for production of high expressing promoters and the production of plants with enhanced expression of nucleic acids wherein combinations of at least two nucleic acid expression enhancing nucleic acids (NEENAs) are functionally linked to said promoters and/or introduced into plants.

### BACKGROUND

Expression levels of transgenes in plants are strongly affected by various external and internal factors resulting in a variable and unpredictable level of transgene expression. Often a high number of transformants must be produced and analyzed to identify lines with desirable expression strength. As transformation and screening of lines with desirable expression strength is costly and labor intensive there is a need for high expression of one or more transgenes in a plant. This problem is especially pronounced, when several genes must be coordinately expressed in a transgenic plant to achieve a specific effect as a plant has to be identified in which each gene is strongly expressed. Alternatively, the endogenous gene expression level of a target gene is often lower than needed in order to become the desired effect. Strong promoters can partially overcome these challenges. Unfortunately, the availability of suitable promoters showing strong expression with the desired specificity is limited. The identification and characterization of new promoters with the respective specificity and strength is, however, a time-consuming process.

In addition to promoters, additional genetic elements and/or motifs that positively affect gene expression can be used to regulate the expression levels of target genes. In particular, enhancers are cis-regulatory DNA elements that regulate transcription programs by recruiting transcription factors and directing them to the promoters of target genes in a cell-type/tissue-specific manner. The expression of a gene can be regulated by one or multiple enhancers (Marand et al 2017; Biochimica and Biophysica Acta 1860 (131-139). Enhancers may be located upstream or downstream of the transcription start site of a certain expressed nucleic acid, may be found within introns and may function at positions 5000 or more nucleotides away from the respective promoter. The unpredictable positions of enhancers, relative to their cognate promoters, has resulted in the identification of only a limited number of enhancers in plant species. WO 2021/048316 A1, WO 2021/069387 A1, WO 2021/110582 A1 and WO 2020/229241 A1 describe the identification of a number of enhancers in plants. While the insertion of strong enhancers in promoters, untranslated regions or introns of endogenous genes using genome editing techniques could allow to significantly increase the expression levels of endogenous genes, this approach is currently impeded by the limited number of enhancers identified in plant species conferring the desired levels of expression.

There remains a need for new gene regulatory elements that can drive strong expression of transgenes as well as endogenous genes in plants.

### SUMMARY

Nucleic acid molecules enhancing expression of functionally linked nucleic acids are in the present disclosure described as “nucleic acid expression enhancing nucleic acids” (NEENA).

In one aspect a method is provided for enhancing expression derived from a plant promoter, comprising functionally linking to the promoter two or more heterologous nucleic acid expression enhancing nucleic acid (NEENA) molecules, wherein a first NEENA molecule and a second NEENA molecule share a nucleic acid sequence identity of at most 80%, said NEENA molecules comprising:
(i) a nucleic acid having a sequence of any one of SEQ ID NOs: 1-41 or their complement, or
(ii) a nucleic acid having at least 90% sequence identity to any one of SEQ ID NOs: 1-41 or their complement, or
(iii) a nucleic acid hybridizing under stringent conditions to any one of SEQ ID NOs: 1-41 or their complement, or
(iv) a functional fragment of 27 or more consecutive bases of a nucleic acid of i) to iii),
wherein the expression derived from said plant promoter is enhanced by 50% or more as compared to said plant promoter functionally linked to only said first or said second NEENA molecule.

In another aspect a method is provided for producing a plant or part thereof with enhanced expression of one or more nucleic acid molecules, as compared to a respective control plant or part thereof, comprising the steps of:
a) introducing into the plant or part thereof two or more heterologous NEENA molecules, wherein a first NEENA molecule and a second NEENA molecule share a nucleic acid sequence identity of at most 80%, said NEENA molecules comprising a nucleic acid molecule as defined above in i) to iv), and
b) functionally linking said two or more NEENA molecules to a promoter and to a nucleic acid molecule being under the control of said promoter, wherein said NEENA molecules are heterologous to said promoter,
wherein said control plant or part thereof comprises said promoter functionally linked only to said first or said second NEENA molecule.

In an embodiment, the first NEENA or the second NEENA comprises: i) a nucleic acid having a sequence of any one of SEQ ID NOs: 1-14 or their complement, or ii) a nucleic acid having at least 90% sequence identity to any one of SEQ ID NOs: 1-14 or their complement, or iii) a nucleic acid hybridizing under stringent conditions to any one of SEQ ID NOs: 1-14 or their complement, or iv) a functional fragment of 27 or more consecutive bases of a nucleic acid of i) to iii).

In another or further embodiment, a first NEENA comprises: i) a nucleic acid having a sequence of any one of SEQ ID NOs: 1-6, or their complement, or ii) a nucleic acid having at least 90% sequence identity to any one of SEQ ID NOs: 1-6 or their complement, or iii) a nucleic acid hybridizing under stringent conditions to any one of SEQ ID NOs: 1-6 or their complement, or iv) a functional fragment of 27 or more consecutive bases of a nucleic acid of i) to iii); and a second NEENA molecule comprises: i) a nucleic acid having the sequence of any one of SEQ ID NOs: 7-14, or their complement, or ii) a nucleic acid having at least 90% sequence identity to any one of SEQ ID NOs: 7-14 or their complement, or iii) a nucleic acid hybridizing under stringent conditions to any one of SEQ ID NOs: 7-14 or their complement, or iv) a functional fragment of 27 or more consecutive bases of a nucleic acid molecule of i) to iii).

In a further embodiment, the method described above comprises the steps of a) introducing said two or more NEENA molecules into a plant or part thereof, and b) integrating said NEENA molecules into the genome of said plant or part thereof whereby said NEENA molecules are functionally linked to an endogenous promoter and to each other, said NEENA molecules being heterologous to said promoter and, optionally, c) regenerating a plant or part thereof comprising said NEENA molecules from said plant or part thereof. In a further embodiment the method the NEENA molecules are integrated into the genome of a plant or part thereof by applying genome editing technologies. In a further embodiment the genome editing technology comprises the introduction of single or double strand breaks near to the position where a NEENA molecule is to be integrated into the genome using nucleic acid guided nucleases, TALEN, homing endonucleases or Zink finger proteins and the introduction of a DNA repair template comprising a NEENA molecule and at its 3′ and 5′ end sequences essentially identical or complementary to the sequences upstream and downstream of the single or double strand break facilitating recombination at the position of the single or double strand break. In a further embodiment the genome editing technology comprises introduction of point mutations in the genome of the plant or part thereof thereby introducing the sequence of a NEENA molecule in the plant genome.

In another further embodiment, the methods described above comprise the steps of a) providing an expression construct comprising two or more NEENA molecules, said NEENA molecules comprising a nucleic acid as defined above in i) to iv) functionally linked to said promoter heterologous to said NEENA molecules and b) integrating said expression construct into the genome of said plant or part thereof and, optionally, c) regenerating a plant or part thereof comprising said one or more expression constructs from said transformed plant or part thereof.

In another further embodiment, the NEENA molecules are functionally linked to the promoter upstream or downstream of the transcriptional start site of a nucleic acid molecule the expression of which is under control of the promoter.

In another further embodiment, the said NEENA molecules are functionally linked to the promoter within the 5′UTR or within an intron of the nucleic acid molecule the expression of which is under control of the promoter.

In another further embodiment, the promoter is a tissue specific, developmental specific or inducible promoter of a nucleic acid molecule the expression of which is under control of the promoter.

In another further embodiment, the NEENA molecules are present in one or more of the configurations selected from the group consisting of head-to-head, head-to-tail, tail-to-head, tail-to-tail, and combinations thereof.

In another further embodiment, the NEENA molecules are separated from each other by a spacer sequence. In a further embodiment, the spacer sequence comprises 1 to 50 nucleotides.

In another further embodiment, a first NEENA molecule comprises a nucleic acid with at least 90% sequence identity to nucleotide 74 to 100 of SEQ ID NOs: 7-8 or a nucleic acid that hybridizes under stringent conditions to nucleotide 74 to 100 of SEQ ID NO: 7-8, and a second NEENA molecule comprises a nucleic acid with at least 90% sequence identity to nucleotide 61 to 101 of SEQ ID NOs: 1-2 or a nucleic acid that hybridizes under stringent conditions to nucleotide 61 to 101 of SEQ ID NOs: 1-2.

In another further embodiment, the promoter is functionally linked to additional copies of the two or more NEENA molecules.

In another aspect a recombinant expression construct is provided comprising two or more NEENA molecules functionally linked to a promoter and one or more expressed nucleic acid molecules, wherein said NEENA molecules are heterologous to said promoter, wherein a first NEENA molecule and a second NEENA molecule share a nucleic acid sequence identity of at most 80%, and wherein said NEENA molecules comprise a nucleic acid as defined above in i) to iv).

In a further aspect, a recombinant expression vector is provided comprising one or more recombinant expression constructs as defined above.

In yet another aspect, a transgenic cell or transgenic plant or part thereof is provided comprising a recombinant expression construct as defined above, a recombinant expression vector as defined above or two or more heterologous NEENA molecules wherein a first NEENA molecule and a second NEENA molecule share a nucleic acid sequence identity of at most 80%, and wherein said NEENA molecules comprise a nucleic acid as defined above i) to iv). In a further embodiment, the transgenic cell, transgenic plant or part thereof is selected or derived from the group consisting of bacteria, fungi, yeasts or plants.

In a further aspect, a transgenic cell culture, transgenic seed, parts or propagation material derived from a transgenic cell or plant or part thereof as defined above are provided comprising a recombinant expression construct as defined above, a recombinant expression vector as defined above or two or more heterologous NEENA molecules wherein a first NEENA molecule and a second NEENA molecule share a nucleic acid sequence identity of at most 80%, and wherein said NEENA molecules comprise a nucleic acid as defined above in i) to iv).

In a final aspect, the use of a transgenic cell culture, transgenic seed, transgenic plant, parts or propagation material derived from a transgenic cell or plant as described above is for the production of foodstuffs, animal feeds, seeds, pharmaceuticals or fine chemical is provided.

### BRIEF DESCRIPTION OF THE DRAWINGS

FIG. 1: Impact of individual nucleotide mutations on the activity of the wheat EN1390 enhancer.

FIG. 2: Impact of individual nucleotide mutations on the activity of the wheat EN3233 enhancer.

FIG. 3: Impact of EN1390 fragments on promoter activity.

FIG. 4: Impact of EN3233 fragments on promoter activity.

FIG. 5: Impact of combinations of selected EN1390 fragments and EN3233 fragments on promoter activity.

FIG. 6: Impact of combinations of selected EN1390 fragments and EN3233 fragments on promoter activity.

### DETAILED DESCRIPTION

The present invention concerns methods to enhance expression of nucleic acids derived from a promoter in plants by operably linking a combination of at least two different nucleic acid expression enhancing nucleic acid (NEENA) molecules to the respective promoter. The inventors have found that operably linking two or more NEENA molecules as described hereunder to a promoter, results in an enhanced expression derived from that promoter where the enhanced expression is higher than what would be expected based on the expression enhancing activity of the individual NEENA molecules.

It is to be understood that this invention is not limited to the particular methodology or protocols. The terminology used herein is for the purpose of describing particular embodiments only and is not intended to limit the scope of the present invention which will be limited only by the appended claims. As used herein, the singular forms “a”, “and” and “the” include plural reference unless the context clearly dictates otherwise. Thus, for example, reference to “a vector” is a reference to one or more vectors and includes equivalents thereof known to those skilled in the art. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20%, preferably 10%, more preferably 5%, even more preferably 2%, most preferably 1% up or down (higher or lower). As used herein, the word “or” means any one member of a particular list and includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. The recitation of numerical ranges by endpoints includes all numbers and fractions subsumed within that range, as well as the recited endpoints. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

The term “nucleic acid expression enhancing nucleic acid” or “NEENA” refers to a sequence and/or a nucleic acid molecule of a specific sequence having the intrinsic property to enhance expression of a nucleic acid under the control of a promoter to which the NEENA is functionally linked. Unlike promoter sequences, the NEENA as such is not able to drive expression. In order to fulfill the function of enhancing expression of a nucleic acid molecule functionally linked to the NEENA, the NEENA itself has to be functionally linked to a promoter. In distinction to enhancer sequences known in the art, the NEENA is acting in cis but not in trans and has to be located close to the transcription start site of the nucleic acid to be expressed.

In one aspect, the invention comprises a method for enhancing expression derived from a plant promoter, comprising functionally linking to the promoter two or more heterologous nucleic acid expression enhancing nucleic acid (NEENA) molecules, wherein a first NEENA molecule and a second NEENA molecule share a nucleic acid sequence identity of at most 80%, more preferably at most 75%, more preferably at most 70%, most preferably at most 65%, when sequence identity is determined over the complete length of the first or over the complete length of the second NEENA, said NEENA molecules comprising:
(i) a nucleic acid having a sequence of any one of SEQ ID NOs: 1-41 or their complement, or
(ii) a nucleic acid having at least 90% sequence identity to any one of SEQ ID NOs: 1-41 or their complement, or
(iii) a nucleic acid hybridizing under stringent conditions to any one of SEQ ID NOs: 1-41 or their complement, or
(iv) a functional fragment of 27 or more consecutive bases of a nucleic acid of i) to iii),
wherein the expression derived from said plant promoter is enhanced by 50% or more as compared to said plant promoter functionally linked to only said first or said second NEENA molecule.

The inventors have found that functionally linking at least two NEENA molecules to a promoter according to the method disclosed above results in an unexpected enhancement of the expression derived from that promoter. This method thus allows to efficiently enhance expression of a desired product when the expression driven by a desired promoter or by a promoter functionally linked only to one or more copies of a single NEENA molecule fails to reach the intended strength.

The term “expression” or “gene expression” includes the transcription of a specific gene or specific genes or specific genetic construct. The term “expression” or “gene expression”, in particular, means the transcription of a gene or genes or genetic construct into structural RNA (rRNA, tRNA) or mRNA with or without subsequent translation of the latter into a protein. The process includes transcription of DNA and processing of the resulting mRNA product. Yet, the term “expression” as used herein may also include the translation of process of an mRNA molecule where a polypeptide is formed. Thus, the term “expression” may include the transcription process alone, the translation process alone, or both processes combined.

“Enhance” or “increase” expression are used equivalently herein and mean that the level of expression of a nucleic acid molecule in a plant, part of a plant or plant cell after applying a method of the present invention is higher than its original level of expression in the plant, part of the plant or plant cell before applying the method or compared to a reference plant lacking a recombinant nucleic acid molecule of the invention. The original level of expression includes the absence of expression and immeasurable expression. For example, the reference plant is comprising the same construct which is only lacking the respective NEENA molecules. As used herein, an “enhancement” or “increase” of the level of an agent such as a protein, mRNA or RNA means that the level is increased relative to a substantially identical plant, part of a plant or plant cell grown under substantially identical conditions, lacking a recombinant nucleic acid molecule of the invention, for example lacking the NEENA molecules, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 75% or more, for example 100% or more, preferably 200% or more, more preferably 5 fold or more, even more preferably 10 fold or more, most preferably 20 fold or more for example 50 fold relative to a cell or organism lacking a recombinant expression construct of the invention.

The enhancement or increase can be determined by methods with which a person skilled in the art is familiar. Thus, the enhancement or increase of the nucleic acid or protein expression can be determined for example by an immunological detection of the protein. Moreover, techniques such as protein assay, fluorescence, Northern hybridization, nuclease protection assay, reverse transcription (quantitative RT-PCR), ELISA (enzyme-linked immuno-sorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its' activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254). As one example for quantifying the activity of a protein, the detection of luciferase activity is described in the Examples below.

The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, ha-lo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Polypeptide: The terms “polypeptide”, “peptide”, “oligopeptide”, “polypeptide”, “gene product”, “expression product” and “protein” are used interchangeably herein to refer to a polymer or oli-gomer of consecutive amino acid residues.

The terms “promoter”, or “promoter sequence” are equivalents and as used herein, refer to a DNA sequence which when operably linked to a nucleotide sequence of interest and is capable of controlling the transcription of the nucleotide sequence of interest into RNA. A “plant promoter” comprises promoters which mediate the expression of a coding sequence segment in plant cells. Accordingly, a plant promoter need not be of plant origin, but may also originate for example from viruses or microorganisms or it may be a synthetic promoter designed by man. Such plant promoters can for example be found in the following public databases GrassPROMDB (https://www.grassius.org/grasspromdb.php), PlantPromDB (http://www.softberry.com/), PPDB (http://ppdb.agr.gifu-u.ac.jp). Promoters listed there may be addressed with the methods of the invention and are herewith included by reference. A promoter is located 5′ (i.e., upstream), proximal to the transcriptional start site of a nucleotide sequence of interest whose transcription it controls and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may be heterologous or homologous to the respective plant.

The term “heterologous” (or exogenous or foreign or recombinant) polynucleotide refers to a polynucleotide that is not native to the host cell; or to a polynucleotide native to the host cell but including structural modifications, e.g., deletions, substitutions, and/or insertions as a result of manipulation of the DNA of the host cell by recombinant DNA techniques to alter the native polynucleotide; or to a polynucleotide native to the host cell whose expression is quantitatively altered as a result of manipulation of the regulatory elements of the polynucleotide by recombinant DNA techniques, e.g., a stronger promoter; or to a polynucleotide native to the host cell, but integrated not within its natural genetic environment as a result of genetic manipulation by recombinant DNA techniques. With respect to two or more polynucleotide sequences or two or more amino acid sequences, the term “heterologous” is used to characterize that the two or more polynucleotide sequences or two or more amino acid sequences do not occur naturally in the specific combination with each other. For example, a promoter operably linked to a heterologous nucleic acid sequence refers to a nucleic acid sequence from a species different from that from which the promoter was derived, or when it originates from the same species, to a nucleic acid sequence which is not naturally associated with the promoter (e.g. a genetically engineered coding sequence or an allele from a different ecotype or variety).

The two or more NEENA molecules may be heterologous to the nucleic acid molecule which is under the control of the promoter to which the NEENAs are functionally linked or it may be heterologous to both the promoter and the nucleic acid molecule under the control of said promoter.

Preferably, the term “heterologous” with respect to a nucleic acid molecule or DNA, for example a NEENA molecule, refers to a nucleic acid molecule which is operably linked to, or is manipulated to become operably linked to, a second nucleic acid molecule, for example a promoter or an additional NEENA molecule, to which it is not operably linked in nature. For example, a NEENA is in its natural environment functionally linked to its native promoter, whereas in the present invention it is linked to another promoter which might be derived from the same organism, a different organism or might be a synthetic promoter such as the SUPER-promoter. It may also mean that a NEENA according to the present invention is linked to its native promoter but the nucleic acid molecule under control of said promoter is heterologous to the promoter comprising its native NEENA. It is in addition to be understood that the promoter and/or the nucleic acid molecule under the control of said promoter functionally linked to two or more NEENAs of the invention are heterologous to said NEENAs as their sequence has been manipulated by for example mutation such as insertions, deletions and the forth so that the natural sequence of the promoter and/or the nucleic acid molecule under control of said promoter is modified and therefore have become heterologous to the NEENAs of the invention. It may also be understood that a NEENA is heterologous to the nucleic acid to which it is functionally linked when the NEENA is functionally linked to its native promoter wherein the position of the NEENA in relation to said promoter is changed.

The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem.

The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

In principle, any promoter is suitable for use in the methods provided herein. These promoters include but are not limited to tissue specific, organ specific, inducible, developmental specific and constitutive promoters.

The two or more NEENAs may be functionally linked to any promoter and will enhance expression of the nucleic acid molecule under control of said promoter. Constitutive promoters to be used in any method of the invention may be derived from plants, for example monocotyledonous or dicotyledonous plants, from bacteria and/or viruses or may be synthetic promoters. Constitutive promoters to be used are for example the Cassava vein mosaic virus-Promoter (Verdaguer B et al. (1996). PMB 31 (6), 1129-39), the Subterrenean Clover Stunt Virus-Promoter (Boevink P, et al. (1995). Virology 207 (2), 354-61), the A. thaliana histone 4A promoter in combination with the histone 3A intron (Chaboute et al. (1984). PMB 8 (2), 179-91), the B. napus P450-dependent fatty acid omega-hydroxylase promoter (WO2016113333), the pAct10s promoter from rice (McElroy et al. (1990). Plant Cell 2 (2), 163-71), the PcUbi-Promoter from P. crispum (WO 2003102198), the ZmUbi-Promoter from Zea mays (Christensen et al (1992). Plant Mol Biol. 18 (4), 675-89), AtNit-promoter from the A. thaliana gene At3g44310 encoding nitrilase 1, the 34S-promoter from figwort mosaic virus (Sanger et al., 1990, PMB 14 (3)), the 35S-promoter from Cauliflower mosaic virus (Odell et al (1985). Nature 313 (6005), 810-2), the nos (Depicker et al (1982). J Mol Appl Genet. 1 (6), 561-73) and ocs-promoter derived from Agrobacterium tumefaciens, the ScBV-promoter (U.S. Pat. No. 5,994,123), the SUPER-promoter (Lee et al. 2007, Plant. Phys. 145), the AtFNR-promoter from the A. thaliana gene At5g66190 encoding the ferredoxin NADH reductase, the ptxA promoter from Pisum sativum (WO2005085450), the AtTPT-promoter from the A. thaliana gene At5g46110 encoding the triose phosphate translocator, the bidirectional AtOASTL-promoter from the A. thaliana genes At4g14880 and At4g14890, the PRO0194 promoter from the A. thaliana gene At1g13440 encoding the glyceraldehyde-3-phosphate dehydrogenase, the PRO0162 promoter from the A. thaliana gene At3g52930 encoding the fructose-bis-phosphate aldolase, the AHAS-promoter (WO2008124495), the CaffeoylCoA-MT promoter and the OsCP12 from rice (WO2006084868) or the pGOS2 promoter from rice (de Pater et al. (1992). Plant J. 2 (6), 837-44).

If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining.

Tissue or developmental specific or inducible promoters to be used in any method of the invention may be derived from plants, for example monocotyledonous or dicotyledonous plants, from bacteria and/or viruses or may be synthetic promoters. Tissue or developmental specific or inducible promoters to be used are for example the seed specific and/or seed-preferential promoters for example the High Molecular Weight Glutenin Bx17 promoter from T. aestivum (Reddy P and Appels R (1993) Theor Appl Genet. 85 (5), 616-24), High Molecular Weight Glutenin 1Dx5 promoter from T. aestivum (Lamacchia et al. (2001) J Exp Bot. 52 (355), 243-50), the plastidic AGPase promoter from T. aestivum (Thorneycroft et al. (2003) Plant Biotechnol J. 1 (4), 259-70), the hordein B1 promoter from Hordeum vulgare (Brandt et al. (1985) Carlsberg Research Communications 50, 333), the SBP-promoter from Vicia faba (WO2000026388), the Unknown Seed Protein-promoter (USP) from Vicia faba (WO2003092362), the napin promoter from Brassica napus (EP0255378), the conlinin-promoter from Linum usitatissmum (WO2001016340), the promoter from the A. thaliana gene At5g01670 encoding the peroxiredoxin like protein (WO2006089950), the promoter of the peroxiredoxin like protein from Linum usitatissmum (WO2006089950), the globulin like protein promoter from Brassica napus (Roh et al., 2014, Journal of the Korean Society for Applied Biological Chemistry 57 (5)), the arcelin5-1 promoter from Phaseolus vulgaris (WO 2012077020), the Zein promoter from Zea mays (Shepherd and Scott Biotechnol Appl Biochem. 2009, 52 (3)), the globulin promoter from Zea mays (Mei et al., 2004, Maydica 49 (4)), the pKG86 promoter from Zea mays (WO 2010122110), the leaf specific ST-LS1 promoter from Solanum tuberosum (Stockhaus et al (1989) EMBO J. 8 (9), 2445-51), the leaf specific thioredoxin promoter from Oryza sativa (Fukuda et al. (2005) Plant Cell Physiol. 46 (11), 1779-86), the root specific or root preferential promoters Pbtg-26D from G. hirsutum (WO2017/025282), PGL4 and 5 from Zea mays (EP1862473) or Pzrp2 from Zea mays (Held et al. (1997) PMG 35 (3), 367-375), the inducible promoters Phpr1 from A. thaliana (Wang et al. (2009) Molecular Plant 2 (1), 191-200), the rd29a promoter from A. thaliana (Yamaguchi-Shinozaki K and Shinozaki K (1994) Plant Cell 6 (2), 251-64), the proteinase inhibitor promoter from Zea mays (Cordero et al (1994) Plant J. 6 (2), 141-50), or the fiber specific or preferential promoters from G. hirsutum as described in WO2012093032, US2013081154, WO2004065571, WO2008083969 or WO2012136788.

The high expression promoters of the invention functionally linked to two or more NEENA molecules may be employed in any plant comprising for example moss, fern, gymnosperm or angiosperm, for example monocotyledonous or dicotyledonous plant. In a preferred embodiment said promoter of the invention functionally linked to two or more NEENA molecules may be employed in monocotyledonous or dicotyledonous plants, preferably crop plant such as corn, soy, canola, cotton, potato, sugar beet, rice, wheat, sorghum, barley, musa, sugarcane, miscanthus and the like. In a preferred embodiment, said promoter which is functionally linked to the NEENAs may be employed in monocotyledonous crop plants such as corn, rice, wheat, sorghum, musa, miscanthus, sugarcane or barley. In a more preferred embodiment, the promoter functionally linked to the NEENAs may be employed in wheat.

A high expressing promoter as used in the application means for example a promoter which is functionally linked to two or more NEENA molecules causing enhanced expression of the promoter in a plant or part thereof, wherein the accumulation of RNA or rate of synthesis of RNA derived from the nucleic acid molecule under the control of the respective promoter functionally linked to two or more NEENA molecules is higher, preferably significantly higher than the expression caused by the same promoter functionally linked to only one of those NEENA molecules. Preferably the amount of RNA of the respective nucleic acid and/or the rate of RNA synthesis and/or the RNA stability in a plant is increased 50% or more, for example 100% or more, preferably 200% or more, more preferably 5 fold or more, even more preferably 10 fold or more, most preferably 20 fold or more for example 50 fold compared to a control plant of same age grown under the same conditions comprising the same promoter the latter being functionally linked to only one of those NEENA molecules.

When used herein, significantly higher refers to statistical significance. The skilled person is aware how to determine statistical significance for example by applying statistical tests, such as the t-test, to the respective data sets. An increase or decrease, for example in gene expression, that is larger than the margin of error inherent to the measurement technique, preferably an increase or decrease by 50% or more of the expression in the control cell or plant, for example 100% or more, preferably 200% or more, more preferably 5 fold or more, even more preferably 10 fold or more, most preferably 20 fold or more for example 50 fold.

Methods for detecting expression conferred by a promoter are known in the art. For example, the promoter may be functionally linked to a marker gene such as beta-Glucuronidase (GUS), green fluorescence protein (GFP) or luciferase (LUC) and the activity of the respective protein encoded by the respective marker gene may be determined in the plant or part thereof. Other methods include but are not limited to measuring the steady state level or synthesis rate of RNA of the nucleic acid molecule controlled by the promoter by methods known in the art such as Northern blot analysis, qPCR, run-on assays or other methods described in the art.

The term “functional linkage” or “operably linked” means that the described components are in a relationship permitting them to function in their intended manner. For example, a regulatory sequence operably linked to a coding sequence is ligated in such a way that expression of the coding sequence is achieved under conditions compatible with the regulatory sequences. Further, with respect to regulatory elements, is to be understood as meaning the sequential arrangement of a regulatory element (e.g. a promoter) with a nucleic acid sequence to be expressed and, if appropriate, further regulatory elements (such as e.g., a terminator or a NEENA) in such a way that each of the regulatory elements can fulfil its intended function to allow, modify, facilitate or otherwise influence expression of said nucleic acid sequence. As a synonym the wording “operable linkage” or “operably linked” may be used. The expression may result, depending on the arrangement of the nucleic acid sequences, in sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, more preferably less than 100 base pairs, most preferably less than 50 base pairs. In a preferred arrangement, the nucleic acid sequence to be transcribed is located behind the promoter in such a way that the transcription start is identical with the desired beginning of the RNA.

Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands; Plant Molecular Biology Labfax (1993) by R. D. D. Croy, published by BIOS Scientific Publications Ltd (UK) and Blackwell Scientific Publications (UK)). However, further sequences, which, for example, act as a linker with specific cleavage sites for restriction enzymes, or as a signal peptide, may also be positioned between the two sequences. Similarly, a linker comprising non-coding nucleotides, herein referred to as “spacer” sequence may be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and can be inserted into a plant genome, for example by transformation.

A skilled person is aware of various methods for functionally linking two or more nucleic acid molecules. Such methods may encompass restriction/ligation, ligase independent cloning, recombineering, recombination or synthesis. Other methods may be employed to functionally link two or more nucleic acid molecules.

The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest. The term “antisense” refers to a nucleotide sequence that is reverse complementary or inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

In an embodiment the nucleic acids of (ii) have at least 90% sequence identity to any one of SEQ ID NOs: 1-41 or their reverse complement, preferably at least 92%, more preferably at least 94%, more preferably at least 95%, at least 96%, at least 97%, at least 99% sequence identity to any one of SEQ ID NOs: 1-41 when sequence identity is determined over the complete length of the respective SEQ ID NOs: 1-41.

“Identity” when used in respect to the comparison of two or more nucleic acid or amino acid molecules means that the sequences of said molecules share a certain degree of sequence similarity, the sequences being partially identical. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases
Seq B: GATCTGA length: 7 bases
   Hence, the shorter sequence is sequence B.

Producing a pairwise global alignment which is showing both sequences over their complete lengths results in:

| | |
|---|---|
| Seq A: | AAGATACTG- |
| | \|\|\| \|\|\| |
| Seq B: | --GAT-CTGA |

The “|” symbol in the alignment indicates identical residues (which means bases for DNA or amino acids for proteins). The number of identical residues is 6. The “-” symbol in the alignment indicates gaps. The number of gaps introduced by alignment within the Seq B is 1. The number of gaps introduced by alignment at borders of Seq B is 2, and at borders of Seq A is 1. The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).
Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

To determine the percent-identity between two nucleic acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters for nucleic acid alignments (gapopen=10.0, gapextend=0.5 and matrix=EDNAFULL).

For nucleic acid sequences, to compare a sequence to any sequence of this invention (SEQ ID NOs: 1-41), the pairwise alignment shall be made over the complete length of the sequence of this invention. Percent identity is then calculated by: %-identity=(identical residues/length of the alignment region which is showing the sequence of this invention over its complete length)*100. For calculating the sequence identities between a first and a second NEENA molecule, a pairwise alignment shall be made over the complete length of both NEENA molecules. Percent identity for the first NEENA molecule is then calculated by: %-identity=(identical residues/length of the alignment region which is showing the sequence of the first NEENA molecule over its complete length)*100. Percent identity for the second NEENA molecule is then calculated by: %-identity=(identical residues/length of the alignment region which is showing the sequence of the second NEENA over its complete length)*100.

In another embodiment, the nucleic acid of (iii) is hybridizing to any one of SEQ ID NOs: 1-41 or their complement under conditions equivalent to hybridization in 7% sodium dodecyl sulfate (SDS), 0.5 M NaPO₄, 1 mM EDTA at 50° C. with washing in 2×SSC, 0.1% SDS at 50° C. or 65° C., preferably 65° C. Preferably, the nucleic acid of (iii) is hybridizing to any one of SEQ ID NOs: 1-41 or their complement under conditions equivalent to hybridization in 7% sodium dodecyl sulfate (SDS), 0.5 M NaPO₄, 1 mM EDTA at 50° C. with washing in 1×SSC, 0.1% SDS at 50° C. or 65° C., preferably 65° C. More preferably, the nucleic acid of (iii) is hybridizing to any one of SEQ ID NOs: 1-41 or their complement under conditions equivalent to hybridization in 7% sodium dodecyl sulfate (SDS), 0.5 M NaPO₄, 1 mM EDTA at 50° C. with washing in 0.1×SSC, 0.1% SDS at 50° C. or 65° C., preferably 65° C.

“Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

The term “antiparallel” refers to two nucleotide sequences paired through hydrogen bonds between complementary base residues with phosphodiester bonds running in the 5′-3′ direction in one nucleotide sequence and in the 3′-5′ direction in the other nucleotide sequence.

The term “hybridization” as defined herein is a process wherein substantially complementary nucleotide sequences anneal to each other. The term hybridization according to this invention means, that hybridization must occur over the complete length of the sequence of the invention. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48:443-453; as defined above). A nucleotide sequence “substantially complementary” to a reference nucleotide sequence hybridizes to the reference nucleotide sequence under low stringency conditions, preferably medium stringency conditions, most preferably high stringency conditions.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may some-times be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduces the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

DNA-DNA hybrids (Meinkoth and Wahl, Anal. Biochem., 138:267-284, 1984):

$${T}_{m} = 81.5 ° C . + 16.6 \times {log [ {Na}^{+} ]}^{a} + 0.41 \times \% [ G / {C}^{b} ] - 500 \times {[ {L}^{c} ]}^{- 1} - 0.61 \times \% formamide$$

### DNA-RNA or RNA-RNA Hybrids:

$${T}_{m} = 79.8 + 18.5 ( {log10 [ {Na}^{+} ]}^{a} ) + 0.58 [ \% G / {C}^{b} ] + {11.8 [ \% G / {C}^{b} ]}^{2} - 820 / {L}^{c}$$

### Oligo-DNA or Oligo-RNA^{d} Hybrids:

For <20 nucleotides: Tₘ=2 (Iₙ)
For 20-35 nucleotides: Tₘ=22+1.46 (Iₙ)
   ^{a}or for other monovalent cation, but only accurate in the 0.01-0.4 M range.
   ^{b}only accurate for % GC in the 30% to 75% range.
   ^{c}L=length of duplex in base pairs.
   ^{d}Oligo, oligonucleotide; Iₙ, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with RNase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridization at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH,

New York or to Current Protocols in Molecular Biology, John Wiley & Sons, N.Y. (1989 and yearly updates).

In another embodiment, the functional fragment of (iv) comprises at least about 27, at least about 30, at least about 35, at least about 41, at least about 50, at least about 60, at least about 70, at least about 80, at least about 90, at least about 100, at least about 110, at least about 120, at least about 130, at least about 140 consecutive bases of the original nucleic acid sequence.

“Fragment”, or “subsequence” as used herein is a portion of a polynucleotide or an amino acid sequence. In some aspects, such fragments or subsequences retain or encode for at least one functional activity of the sequence to which it is related.

The term “functional fragment” refers to any nucleic acid sequence which comprises merely a part of the full-length nucleic acid sequence, but still has the same or similar activity and/or function. A “functional fragment” of a nucleic acid having expression enhancing activity denotes a nucleic acid comprising a stretch of the nucleic acid sequence of any one of SEQ ID NOs: 1 to 41 or their complement, or of the nucleic acid having at least 95% sequence identity to any one of SEQ ID NOs: 1 to 41 or their complement, or of the nucleic acid hybridizing under stringent conditions to any one of SEQ ID NOs: 1 to 41 or their complement, which still exerts the desired function, i.e. which has 50% or more, preferably 60% or more, more preferably 70% or more, even more preferably 80% or more, 85% or more or 90% or more, most preferred 95% or more of the expression enhancing activity as the corresponding original nucleic acid sequence.

In another aspect, the invention provides a method for producing a plant or part thereof with enhanced expression of one or more nucleic acid molecules, as compared to a respective control plant or part thereof, comprising the steps of:
a) introducing into the plant or part thereof two or more heterologous NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA, said NEENA molecules comprising a nucleic acid molecule as defined above in i) to iv), and
b) functionally linking said two or more NEENA molecules to a promoter and to a nucleic acid molecule being under the control of said promoter, wherein said NEENA molecules are heterologous to said promoter,
wherein said control plant or part thereof comprises said promoter functionally linked only to said first or said second NEENA molecule.

A “plant” is generally understood as meaning any eukaryotic single or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes whole plants, ancestors and progeny of the plants and plant parts, including shoots, stems, leaves, roots (including tubers), propagation material (such as seeds or microspores), flowers, seedlings and mature plants, tissues and organs (mature plants and seedlings) and protoplasts and their derived parts.

The term “plant” also refers to plant cells, cell cultures such as suspension cultures, callus tissue, embryos, meristematic regions, gametophytes, sporophytes, pollen and microspores. Hence, when the term “plant” is used herein, the term is intended to encompass “a plant, part thereof, or plant cell”. The term “plant cell” may encompass a non-propagating plant cell.

Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants.

The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

A plant exhibiting enhanced expression of a nucleic acid molecule as used herein refers to a plant having a higher, preferably statistically significant higher expression of a nucleic acid molecule compared to a control plant grown under the same conditions without the respective two or more NEENAs functionally linked to the respective nucleic acid molecule. Such control plant may be a wild-type plant or a transgenic plant comprising the same promoter controlling the same gene as in the plant of the invention wherein the promoter is functionally linked to only one of those NEENA molecules. The term “wild-type”, “natural” or “natural origin”, as used herein, refers to an organism, polypeptide, or nucleic acid sequence which is naturally occurring or, in the case of a polypeptide or a nucleic acid, which is available in at least one naturally occurring organism which is not changed, mutated, or otherwise manipulated by man.

Producing a plant as used herein comprises methods for stable transformation such as introducing a recombinant DNA construct into a plant or part thereof by means of Agrobacterium mediated transformation, protoplast transformation, particle bombardment or the like and optionally subsequent regeneration of a transgenic plant. Producing a plant, as used herein, also comprises methods for transient transformation of a plant or part thereof such as viral infection or Agrobacterium infiltration. A skilled person is aware of further methods for stable and/or transient transformation of a plant or part thereof.

Approaches such as breeding methods, protoplast fusion or recombination techniques using a donor DNA might also be employed for production of a plant of the invention and are covered herewith. For example, a single strand break (nick) or a double strand break may be introduced into the genome of a plant using recombinant technologies known in the art such as TALEN (WO12138939, WO12138927); Zink finger proteins (WO02057293, WO05084190), homing endonucleases (WO11104382, WO14199358) or nucleic acid guided nucleases such as AGO, Cas9 or Cas12 (WO13141680, WO13176772, WO14093595, WO15157534 or WO16205711). Together with the introduction of such single or double-strand break inducing agents, one or more donor DNA (WO13176772, WO14089290) may be introduced into the plant or part thereof comprising one or more NEENA molecules flanked by nucleic acid molecules comprising sequences essentially identical or essentially complementary to the regions adjacent to the nick or double strand break thereby facilitating homologous recombination and introducing the NEENA molecule into the genome of the plant or part thereof.

Further, the sequence of two or more NEENAs of the invention may be introduced into the genome and functionally linked to the respective heterologous promoter by introducing into the genome a series of point mutations using technologies such as deaminases (WO0058480, WO18027078) and the like which may be directed to a specific region in the genome of a plant or part thereof by fusing the mutating polypeptide portion e.g. a deaminase or glycosidase to a DNA binding polypeptide such as, for example a TALEN, a Zinc finger protein, a homing endonuclease or an RNA guided nuclease, nickase or inactivated nuclease such as Cas9 or Cas12, as described in WO15089406, US2017321210, WO15133554 or WO17070632. By application of these methods, the NEENA sequences are introduced into the genome without introduction of a heterologous molecule, the NEENA sequences replacing other sequences in the genome. Such technologies are encompassed by the term “integrate”, “integrating”, “introduce” or “introducing” a NEENA sequence or a NEENA molecule into the genome and functionally linking such sequences and/or molecules to a heterologous promoter.

The methods of the invention may be applied to any plant, for example gymnosperm or angiosperm, preferably angiosperm, for example dicotyledonous or monocotyledonous plants, preferably monocotyledonous plants. Preferred monocotyledonous plants are for example corn, wheat, rice, barley, sorghum, musa, sugarcane, miscanthus and brachypodium, especially preferred monocotyledonous plants are corn, wheat and rice, most preferred is wheat. Preferred dicotyledonous plants are for example soy, rape seed, canola, linseed, cotton, potato, sugar beet, Tagetes and Arabidopsis, especially preferred dicotyledonous plants are soy, rape seed, canola and potato.

In a further embodiment of the invention, the methods as defined above comprise a first NEENA or a second NEENA molecule comprising:
i) a nucleic acid having a sequence of any one of SEQ ID NOs: 1-14 or their complement, or
ii) a nucleic acid having at least 90% sequence identity to any one of SEQ ID NOs: 1-14 or their complement, or
iii) a nucleic acid hybridizing under stringent conditions to any one of SEQ ID NOs: 1-14 or their complement, or
iv) a functional fragment of 27 or more consecutive bases of a nucleic acid of i) to iii).

In a further preferred embodiment, the methods as defined above comprise a first NEENA molecule comprising:
i) a nucleic acid having a sequence of any one of SEQ ID NOs: 1-6 or their complement, or
ii) a nucleic acid having at least 90% sequence identity to any one of SEQ ID NOs: 1-6 or their complement, or
iii) a nucleic acid hybridizing under stringent conditions to any one of SEQ ID NOs: 1-6 or their complement, or
iv) a functional fragment of 27 or more consecutive bases of a nucleic acid of i) to iii);
and a second NEENA molecule comprising:
i) a nucleic acid having the sequence of any one of SEQ ID NOs: 7-14 or their complement, or
ii) a nucleic acid having at least 90% sequence identity to any one of SEQ ID NOs: 7-14 or their complement, or
iii) a nucleic acid hybridizing under stringent conditions to any one of SEQ ID NOs: 7-14 or their complement, or
iv) a functional fragment of 27 or more consecutive bases of a nucleic acid molecule of i) to iii).

In an embodiment of the invention, the methods as defined above comprise the steps of
a) introducing two or more NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA, and wherein the NEENA molecules comprise a nucleic acid molecule as described above in i) to iv) into a plant or part thereof, and
b) integrating the NEENA molecules into the genome of the plant or part thereof whereby the NEENA molecules are functionally linked to an endogenous promoter and to each other, the NEENA molecules being heterologous to the promoter and, optionally,
c) regenerating a plant or part thereof comprising the NEENA molecules from said plant or part thereof.

The two or more NEENA molecules may be introduced into the plant or part thereof by means of particle bombardment, protoplast electroporation, virus infection, Agrobacterium mediated transformation, CRISPR/Cas or any other approach known in the art. The NEENA molecules may be introduced integrated for example into a plasmid or viral DNA or viral RNA or a donor DNA in a CRISPR/Cas approach. The NEENA molecules may also be comprised on a BAC, YAC or artificial chromosome prior to introduction into the plant or part of the plant. Each NEENA molecule may also be introduced as a linear nucleic acid molecule comprising the respective NEENA sequence wherein additional sequences may be present adjacent to the NEENA sequence on the nucleic acid molecule. These sequences neighboring the NEENA sequences may be from about 20 bp, for example 20 bp to several hundred base pairs, for example 100 bp or more and may facilitate integration into the genome for example by homologous recombination. Any other method for genome integration may be employed, be it targeted integration approaches, such as homologous recombination or random integration approaches, such as illegitimate recombination.

The nucleic acid molecule to which the NEENA molecules may be functionally linked may be any nucleic acid, preferably any expressed nucleic acid molecule. The nucleic acid molecule may be a protein coding nucleic acid molecule or a non-coding molecule such as antisense RNA, rRNA, tRNA, miRNA, ta-siRNA, siRNA, dsRNA, snRNA, snoRNA or any other noncoding RNA known in the art. The nucleic acid molecule functionally linked to two or more NEENA molecules may be an endogenous or heterologous nucleic acid molecule.

An “endogenous” nucleotide sequence refers to a nucleotide sequence, which is present in the genome of an untransformed plant cell. The term “heterologous” with respect to a nucleic acid molecule or DNA refers to a nucleic acid molecule which is operably linked to, or is manipulated to become operably linked to, a second nucleic acid molecule, e.g. a promoter or NEENA molecule to which it is not operably linked in nature, e.g. in the genome of a WT plant, or to which it is operably linked at a different location or position in nature, e.g. in the genome of a WT plant. A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

In a further embodiment of the methods of the invention the two or more NEENA molecules or NEENA sequences are integrated into the genome of a plant or part thereof by applying genome editing technologies. In a further preferred embodiment, the genome editing technology comprises the introduction of single or double strand breaks near to the position where a NEENA molecule or sequence is to be integrated into the genome using nucleic acid guided nucleases, for example AGO, Cas9 or Cas12 nucleases, TALEN, homing endonucleases or Zinc finger proteins and further the introduction of a DNA repair template comprising the NEENA molecule or sequence and at its 3′ and 5′ end sequences essentially identical or essentially complementary to the sequences upstream and/or downstream of the single or double strand break facilitating recombination at the position of the single or double strand break. Preferably, the essentially identical or essentially complementary sequences are each individually at least 1000, at least 500 bases, at least 450 bases, at least 400 bases, at least 350 bases, at least 300 bases, at least 250 bases, at least 200 bases, at least 150 bases, at least 100 bases or at least 50 bases long. Preferably, the identity or complementarity of the sequences is at least 50%, at least 60%, at least 70% at least 75%, at least 80%, at least 85%, at least 90%, at least 95%, at least 96%, at least 97%, at least 98%, at least 98 or at least 99% identical or complementary to the respective genomic region with which they recombine.

In another further preferred embodiment, the genome editing technology comprises introduction of point mutations in the genome of the plant or part thereof thereby introducing the sequence of a NEENA molecule in the plant genome. This can for example be achieved by introducing DNA binding proteins, for example Zinc finger proteins, TALE proteins or a nucleic acid guided nuclease, for example Cas9, Cas12 (Cpf1) or AGO functionally bound to a cytidine deaminase (WO17070633) or adenine deaminase (WO18027078).

In another embodiment of the invention, the methods described above comprise the steps of:
a) providing an expression construct comprising two or more NEENA molecules functionally linked to a promoter, the latter being heterologous to said two or more NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA, and wherein the NEENA molecules comprise a nucleic acid molecule as described above in i) to iv), and
b) integrating the expression construct into the genome of a plant or part thereof and, optionally,
c) regenerating a plant or part thereof comprising said one or more expression construct from said transformed plant or part thereof.

The NEENA molecules may be heterologous to the nucleic acid molecule which is under the control of the promoter to which the NEENA is functionally linked or they may be heterologous to both the promoter and the nucleic acid molecule under the control of that promoter.

The expression construct may be integrated into the genome of the respective plant with any method known in the art. The integration may be random using methods such as particle bombardment or Agrobacterium mediated transformation or CRISPR/Cas applications. In a preferred embodiment, the integration is via targeted integration for example by homologous recombination. The latter method would allow integrating the expression construct comprising a high expression promoter functionally linked to two or more NEENA molecules into a favorable genome region. Favorable genome regions are for example genome regions known to comprise genes that are highly expressed for example in seeds and hence may increase expression derived from said expression construct compared to a genome region which shows no transcriptional activity.

In another preferred embodiment, the two or more NEENA molecules are functionally linked close to the transcription start site of the heterologous nucleic acid molecule. Close to the transcription start site as meant herein comprises functionally linking two or more NEENA molecules to a promoter 5000 bp or less, 4000 bp or less, 3000 or less, 2500 bp or less, preferentially 2000 bp or less, more preferred 1500 bp or less, even more preferred 1000 bp or less and most preferred 500 bp or less away from the transcription start site of said heterologous nucleic acid molecule. It is to be understood that the NEENA molecules or sequences may be integrated upstream or downstream in the respective distance from the transcription start site of the respective promoter. Hence, the two or more NEENA molecules may be included in the primary transcript of the respective heterologous nucleic acid under control of the promoter the two or more NEENA molecules are functionally linked to, or they may be integrated within the promoter. If the NEENA molecules are integrated downstream of the transcription start site of the respective promoter, the integration site is preferably in the 5′ UTR, the 3′ UTR or intron of the heterologous nucleic acid the expression of which is under the control of that promoter, most preferentially it is integrated in the first intron of the respective heterologous nucleic acid. Preferentially the two or more NEENA molecules or sequences are integrated in the promoter, the 5′ UTR or the first intron or, similarly, the NEENA molecules are replacing a part in the promoter, the 5′UTR or the first intron.

The term “intron” as used herein, refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are, in addition to other sequences, essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron are comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. The branchpoint sequence is important in splicing and splice-site selection in plants. The branchpoint sequence is usually located 10-60 nucleotides upstream of the 3′ splice-site.

As used herein, the term “coding region”, when used in reference to a structural gene, refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

The term “primary transcript” as used herein refers to a premature RNA transcript of a gene. A “primary transcript”, for example, still comprises introns and/or is not yet comprising a polyA tail or a cap structure and/or is missing other modifications necessary for its correct function as a transcript such as, for example, trimming or editing.

In another aspect of the invention wherein said two or more NEENA molecules are linked to the 7A trehalose-6-phosphate phosphatase (T6PP) gene (WO2018113702, SEQ ID NO: 42), the NEENA molecules may be inserted at about 200 bp, at about 397 bp, at about 676 bp, or at about 1000 bp upstream of the translation start codon. Said one or more NEENA may be inserted into the 7A trehalose-6-phosphate phosphatase (T6PP) gene at a position between 150 and 250 bp, between 350 and 450 bp, between 620 and 720 bp or between 950 and 1000 bp upstream of the translation start codon.

In an embodiment of the invention, the individual NEENA molecules are present in any orientation relative to each other. Accordingly, the two or more NEENA molecules may be present in one or more of the configurations selected from the group consisting of: head-to-head, head-to-tail, tail-to-head, tail-to-tail, and combinations thereof when more than two NEENA molecules are functionally linked to the promoter of a nucleic acid molecule the expression of which is under control of that promoter. In certain further embodiments, additional copies of one or more NEENA molecules are functionally linked to the promoter. The NEENA molecules referred to in the methods described herein may be contiguous with reference to each other or may be separated by a spacer sequence, which may comprise about 1 to 50 nucleotides. Preferably, the spacer sequence comprises about 1 to 40 nucleotides, more preferably about 1 to 30, even more preferably about 1 to 20 nucleotides, most preferably about 1 to 10 nucleotides. The spacer sequence may comprise one or more nucleotides which are naturally flanking the selected NEENA molecule or functional fragment thereof.

In a further embodiment, the methods described above comprise
a first NEENA molecule comprising a nucleic acid with at least 90% sequence identity to nucleotide 74 to 100 of SEQ ID NOs: 7-8 or a nucleic acid that hybridizes under stringent conditions to nucleotide 74 to 100 of SEQ ID NOs: 7-8, and
a second NEENA molecule comprising a nucleic acid with at least 90% sequence identity to nucleotide 61 to 101 of SEQ ID NOs: 1-2 or a nucleic acid that hybridizes under stringent conditions to nucleotide 61 to 101 of SEQ ID NOs: 1-2.

In certain further embodiments of the invention, additional copies of one or more NEENA molecules are functionally linked to the promoter of a nucleic acid molecule the expression of which is under control of that promoter.

In another embodiment of the invention, an isolated nucleic acid is provided comprising at least two or more heterologous NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA and wherein said NEENA molecules comprise a nucleic acid as described above in i) to iv). The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. The isolated nucleic acid sequence may be present in single-stranded or double-stranded form. When an isolated nucleic acid sequence is to be used to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded). As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

A further embodiment of the invention comprises a recombinant expression construct comprising two or more NEENA molecules functionally linked to a promoter and one or more expressed nucleic acid molecules, wherein said NEENA molecules are heterologous to said promoter, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second, and wherein said NEENA molecules comprise a nucleic acid as defined above in i) to iv).

“Expression construct” as used herein refers to a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is-optionally-operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In the case of a plant, the promoter can also be specific to a particular tissue or organ or stage of development.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

The term “recombinant” with respect to nucleic acid molecules refers to nucleic acid molecules produced by recombinant DNA techniques. Recombinant nucleic acid molecules may also comprise molecules, which as such do not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

The recombinant expression construct may further comprise one or more promoters to which the two or more NEENA molecules are functionally linked and optionally one or more expressed nucleic acid molecules the latter being heterologous to said two or more NEENA molecules. The NEENA molecules may be heterologous to the nucleic acid molecule which is under the control of the promoter to which the NEENA molecules are functionally linked or they may be heterologous to both the promoter and the nucleic acid molecule under the control of that promoter.

The expression construct may comprise one or more, for example two or more, for example 5 or more, such as 10 or more combinations of promoters functionally linked to two or more NEENA molecules and a nucleic acid molecule to be expressed heterologous to the respective NEENA molecules. The expression construct may also further comprise additional promoters which are not functionally linked to two or more NEENA molecules and which control the expression of nucleic acid molecules homologous or heterologous to the respective promoter.

Another embodiment of the invention provides a recombinant expression vector comprising one or more recombinant expression constructs as defined above. A multitude of expression vectors that may be used in the present invention are known to a skilled person. Methods for introducing such a vector comprising such an expression construct, comprising for example a promoter functionally linked to two or more NEENA molecules and optionally other elements such as a terminator, into the genome of a plant and for recovering transgenic plants from a transformed cell are also well known in the art. Depending on the method used for the transformation of a plant or part thereof the entire vector might be integrated into the genome of said plant or part thereof or certain components of the vector might be integrated into the genome, such as, for example a T-DNA.

As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

The term “gene” as used herein refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used here-in is intended to mean a DNA sequence that is transcribed into mRNA which is then translated into a sequence of amino acids characteristic of a specific polypeptide.

The term “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

A transgenic cell or transgenic plant or part thereof comprising two or more heterologous NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA and wherein the NEENA comprises a nucleic acid as defined above in i) to iv), is also enclosed in this invention. A NEENA is to be understood as being heterologous to the plant if it is synthetic, derived from another organism or the same organism but its natural genomic localization is modified compared to a control plant, for example a wild-type plant. It is to be understood, that a modified genomic localization means the NEENA is located on another chromosome or on the same chromosome but 10 kb or more, for example 10 kb, preferably 5 kb or more, for example 5 kb, more preferably 1000 bp or more, for example 1000 bp, even more preferably 500 bp or more, for example 500 bp, especially preferably 100 bp or more, for example 100 bp, most preferably 10 bp or more, for example 10 bp dislocated from its natural genomic localization in a wild-type plant.

The term “transgene” as used herein, refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence. The term “transgenic” when referring to an organism means transformed, preferably stably transformed, with a recombinant DNA molecule that preferably comprises a suitable promoter operatively linked to a DNA sequence of interest.

A transgenic cell or transgenic plant or part thereof comprising a recombinant expression vector as defined above or a recombinant expression construct as defined above is a further embodiment of the invention. The transgenic cell, transgenic plant or part thereof may be selected from the group consisting of bacteria, fungi, yeasts or plant, insect or mammalian cells or plants. Preferably, the transgenic cells are bacteria, fungi, yeasts or plant cells. Preferred bacteria are Enterobacteria such as E. coli and bacteria of the genus Agrobacteria, for example Agrobacterium tumefaciens and Agrobacterium rhizogenes. Preferred plants are monocotyledonous or dicotyledonous plants for example monocotyledonous or dicotyledonous crop plants such as corn, soy, canola, cotton, potato, sugar beet, rice, wheat, sorghum, barley, miscanthus, musa, sugarcane and the like. Preferred crop plants are corn, rice, wheat, soy, canola, cotton or potato. Especially preferred dicotyledonous crop plants are soy, canola, cotton or potato. Especially preferred monocotyledonous crop plants are corn, wheat and rice. Most preferred monocotyledonous crop is wheat.

In another embodiment, the invention provides a transgenic cell culture, transgenic seed, parts or propagation material derived from a transgenic cell or plant or part thereof as defined above comprising two or more heterologous NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA and wherein the NEENA molecules comprise a nucleic acid as described above in i) to iv). In yet another embodiment, a transgenic cell culture, transgenic seed, parts or propagation material derived from a transgenic cell or plant or part thereof as defined above comprising a recombinant expression construct or a recombinant expression vector as described above are other embodiments of the invention. Transgenic parts or propagation material as meant herein comprise all tissues and organs, for example leaf, stem and fruit as well as material that is useful for propagation and/or regeneration of plants such as cuttings, scions, layers, branches or shoots comprising the respective NEENA molecules, recombinant expression construct or recombinant vector.

Use of a transgenic cell culture, transgenic seed, parts or propagation material derived from a transgenic cell or transgenic plant or part thereof as defined above for the production of foodstuffs, animal feeds, seeds, pharmaceuticals or fine chemicals is also enclosed in this invention.

A further embodiment of the invention is the use of two or more NEENA molecules, wherein a first NEENA molecule and a second NEENA molecule share a nucleic acid sequence identity of at most 80%, more preferably at most 75%, more preferably at most 70%, most preferably at most 65% when sequence identity is determined over the complete length of the first or over the complete length of the second NEENA. and wherein the NEENA molecules comprise a nucleic acid as described above in i) to iv) or the recombinant construct or recombinant vector as defined above for enhancing expression derived from a plant promoter in plants or parts thereof.

The application at hand provides combinations of gene expression enhancing nucleic acid molecules and constructs comprising one or more promoter functionally linked to two or more NEENA molecules. Additionally, use of such gene expression enhancing nucleic acid molecules and expression constructs, expression vectors, transgenic plants or parts thereof and transgenic cells comprising such gene expression enhancing nucleic acid molecules are provided.

### FIGURES

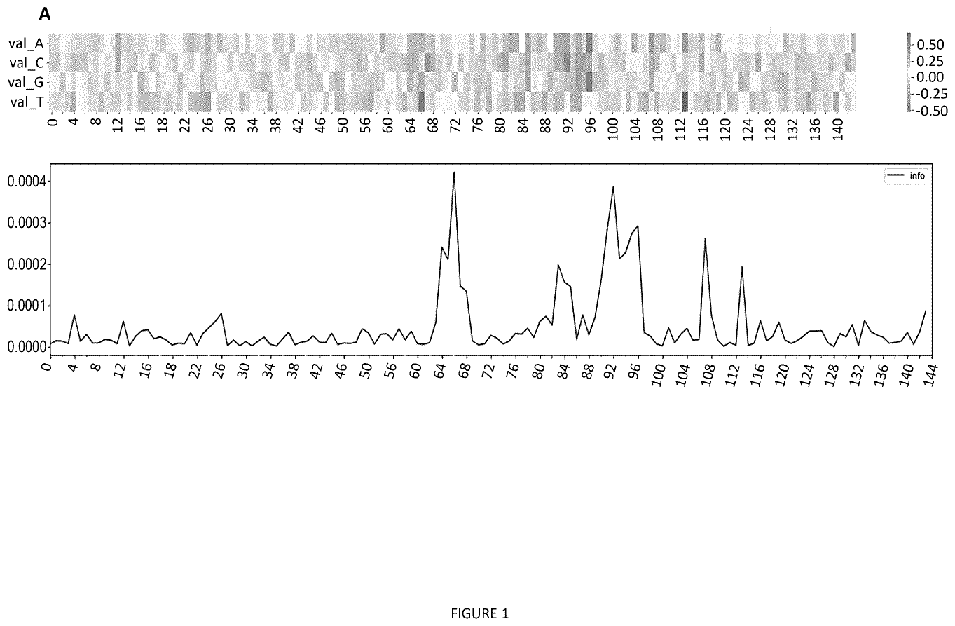

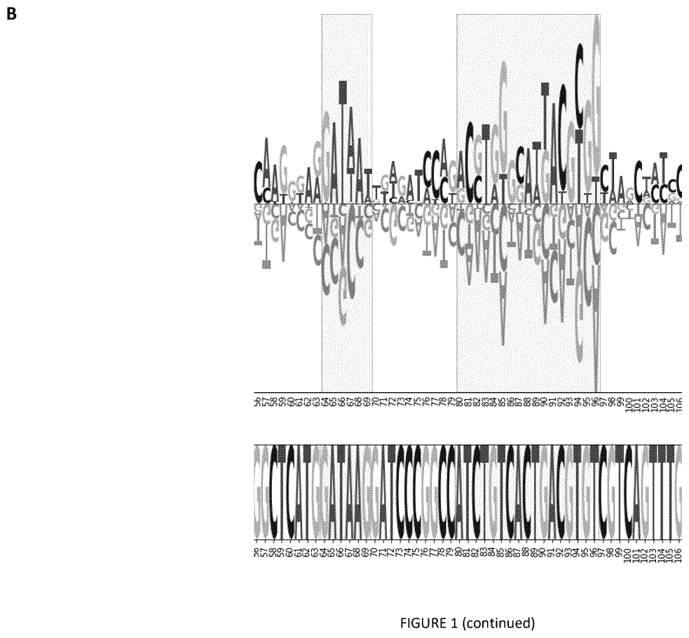

FIG. 1: Impact of individual nucleotide mutations on the activity of the wheat EN1390 enhancer fragment in transiently transformed wheat protoplasts.

The numbers below the graphs show the nucleotide (nt) positions with position 0 corresponding to nt 1 of the enhancer sequence. (A) Blue line graph (above) and z-scaled normalized ER quantitative model graph (below) based on the mutual information of the nucleotide exchange. (B) Logo representing the importance of each nucleotide mutation in free energy (delta delta G) for the region containing the identified important motifs (nt 65-70 and 81-97) putatively explaining the enhancer activity (highlighted). The logo below is representing the initial sequence.

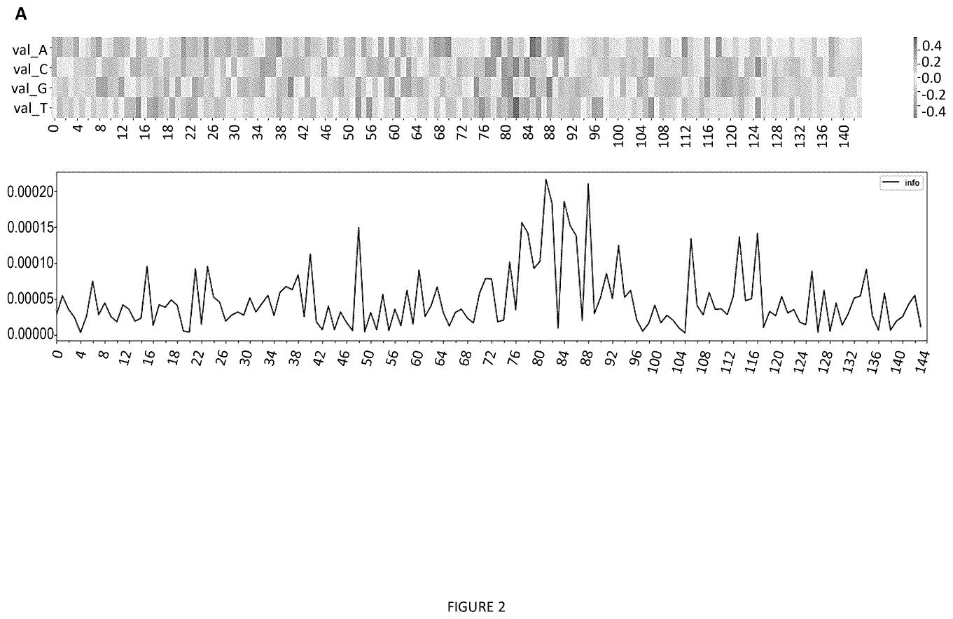

FIG. 2: Impact of individual nucleotide mutations on the activity of the wheat EN3233 enhancer fragment in transiently transformed wheat protoplasts.

The numbers below the graphs show the nucleotide positions with position 0 corresponding to nt 1 of the enhancer sequence. (A) Blue line graph (above) and z-scaled normalized ER quantitative model graph (below) based on the mutual information of the nucleotide exchange. (B) Logo representing the importance of each nucleotide mutation in free energy (delta delta G) for the region containing the identified important motifs (nt 79-94 and 106-126) putatively explaining the enhancer activity (highlighted). The logo below is representing the initial sequence.

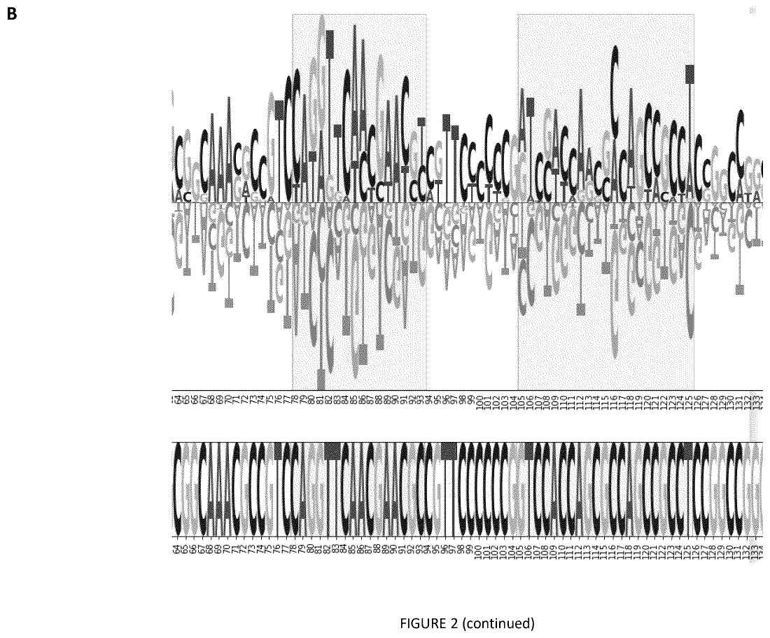

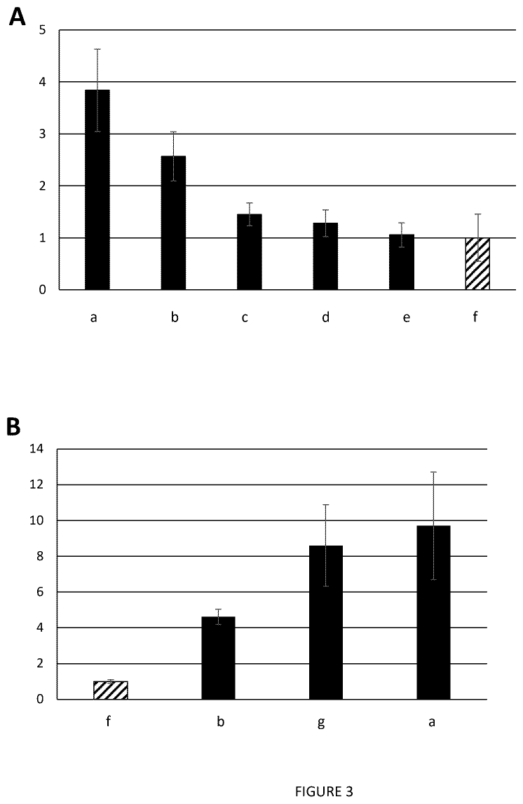

FIG. 3: Impact of EN1390 fragments on the activity of the minimal CaMV 35S promoter (A) or the wheat T6PP promoter (B) in transiently transformed wheat protoplasts.

The vertical axis shows the relative promoter activity. The horizontal axis legend indicates the EN1390 enhancer fragments tested, with the following nucleotide positions: a: nt 1-144, b: nt 61-101, c: nt 77-101, d: nt 81-97, e: nt 61-76, f: none, g: nt 61-101 of the EN1390 predicted optimal sequence. GUS activities were corrected for variation in protoplast transfection efficiency using the luciferase activities of a co-introduced pKA63 plasmid. Activity of the promoter without enhancer was set at 1.

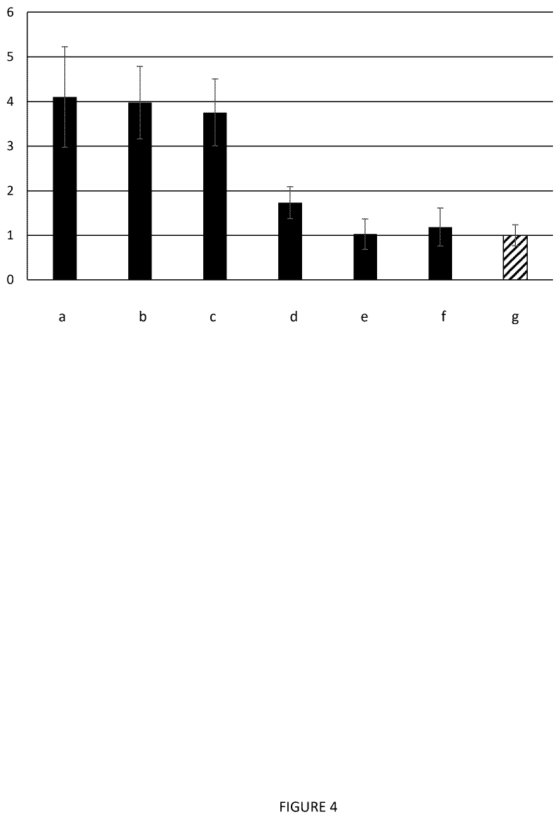

FIG. 4: Impact of EN3233 fragments on activity of the minimal CaMV 35S promoter in transiently transformed wheat protoplasts.

The vertical axis shows the relative promoter activity. The horizontal axis legend indicates the EN3233 enhancer fragments tested, with the following nucleotide positions: a: nt 1-144, b: nt 74-132, c: nt 74-100, d: nt 102-132, e: nt 79-97, f: 106-126, g: none. GUS activities were corrected for variation in protoplast transfection efficiency using the luciferase activities of a co-introduced pKA63 plasmid. Activity of the promoter without enhancer was set at 1.

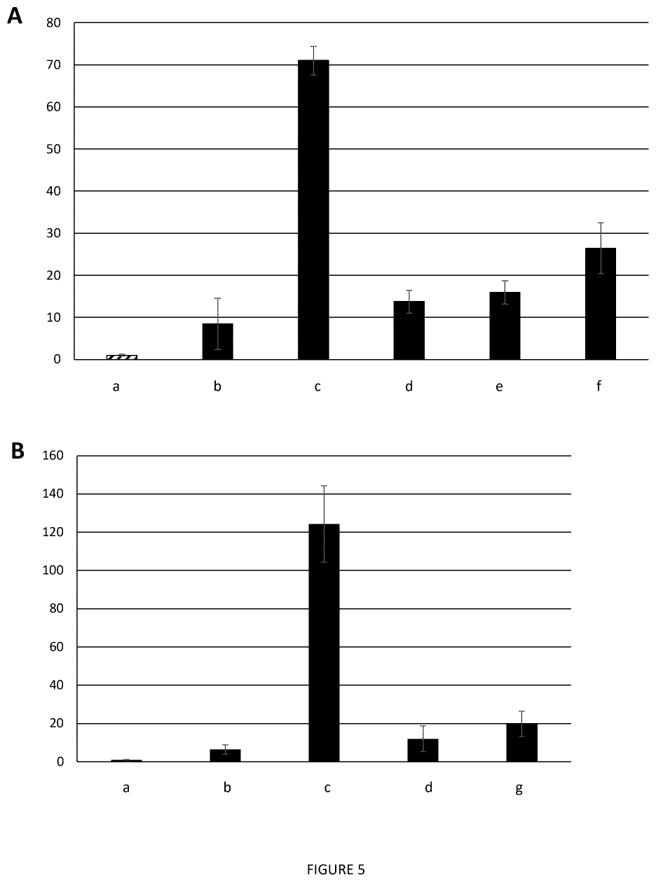

FIG. 5: Impact of EN1390 and EN3233 fragments on activity of the wheat T6PP promoter in transiently transformed wheat protoplasts.

The vertical axis shows the relative promoter activity. The horizontal axis legend indicates the enhancer fragments tested, with the number of the enhancer(s) from which the fragments are derived and the nucleotide positions of the selected fragments in the original enhancer (see Table 3 for corresponding sequences) being as follows: a: none, b: nt 74-100 of EN3233, c: nt 61-101 of EN1390 and nt 74-100 of EN3233, d: nt 61-101 of EN1390, e: nt 1-144 of EN1390, f: nt 1-144 of EN3233, g: nt 61-101 of EN1390 predicted optimal sequence. GUS activities were corrected for variation in protoplast transfection efficiency using the luciferase activities of a co-introduced pKA63 plasmid. Activity of the promoter without enhancer was set at 1. A and B represent two independent experiments.

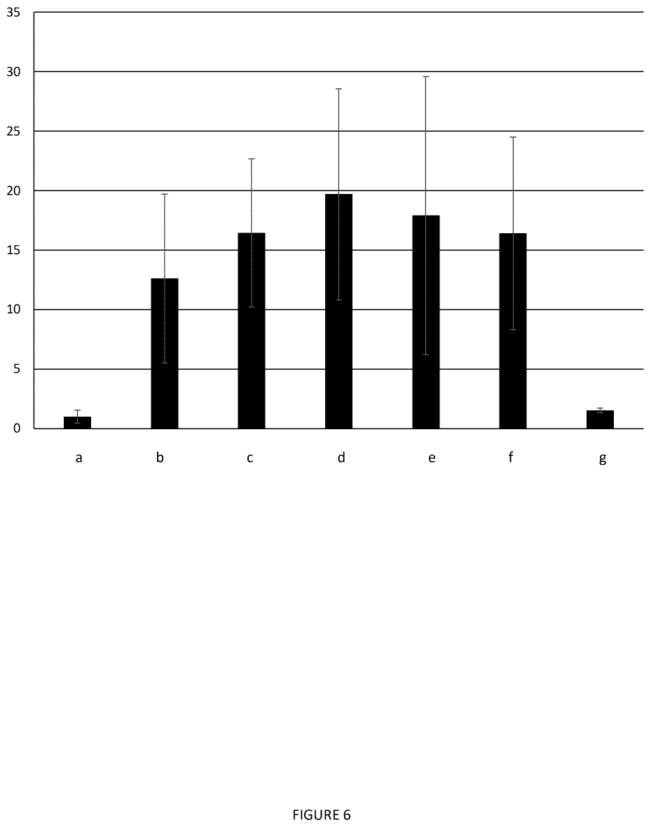

FIG. 6: Impact of EN1390 and EN3233 fragments on activity of the wheat T6PP promoter in transiently transformed wheat protoplasts.

The vertical axis shows the relative promoter activity. The horizontal axis legend indicates the enhancer fragments tested, with the enhancer(s) from which the fragments are derived and the nucleotide positions of the selected fragments in the original enhancer being as follows: a: nt 74-100 of EN3233, b: nt61-101 of EN1390 and nt 74-100 of EN3233, c: nt 74-100 of EN3233 and nt 61-101 of EN1390, d: nt 61-101 of EN1390 and nt 100-74 of EN 3233, e: nt 101-61 of EN1390 and nt 74-100 of EN3233, f: nt 61-101 of EN1390 predicted optimal sequence and nt 74-100 of EN3233, g: nt 61-100 of EN1390. When positions run from high to low, the fragment was present in the reverse orientation. See Table 3 for the corresponding sequences. GUS activities were corrected for variation in protoplast transfection efficiency using the luciferase activities of a co-introduced pKA63 plasmid. Activity of the promoter with only the EN3233 enhancer fragment nt 1-144 inserted was set at 1.

### SEQUENCE LISTING

The sequence listing contained in the file named “220312WO01_Std26.xml”, which is 63 kilobytes (size as measured in Microsoft Windows®), contains 46 sequences SEQ ID NO: 1 through SEQ ID NO: 46 is filed herewith by electronic submission and is incorporated by reference herein.

In the description and examples, reference is made to the following sequences:
SEQ ID NO: 1: Nucleotide sequence of NEENA EN1390
SEQ ID NO: 2: Nucleotide sequence of NEENA EN1390mut
SEQ ID NO: 3: Nucleotide sequence of NEENA EN1390 (nt 81-97)
SEQ ID NO: 4: Nucleotide sequence of NEENA EN1390mut (nt 81-97)
SEQ ID NO: 5: Nucleotide sequence of NEENA EN1390 (nt 61-101)
SEQ ID NO: 6: Nucleotide sequence of NEENA EN1390mut (nt 61-101)
SEQ ID NO: 7: Nucleotide sequence of NEENA EN3233
SEQ ID NO: 8: Nucleotide sequence of NEENA EN3233mut
SEQ ID NO: 9: Nucleotide sequence of NEENA EN3233 (nt 79-94)
SEQ ID NO: 10: Nucleotide sequence of NEENA EN3233mut (nt 79-94)
SEQ ID NO: 11: Nucleotide sequence of NEENA EN3233 (nt 74-100)
SEQ ID NO: 12: Nucleotide sequence of NEENA EN3233mut (nt 74-100)
SEQ ID NO: 13: Nucleotide sequence of NEENA EN3233 (nt 106-126)
SEQ ID NO: 14: Nucleotide sequence of NEENA EN3233mut (nt 106-126)
SEQ ID NO: 15: Nucleotide sequence of NEENA EN5458
SEQ ID NO: 16: Nucleotide sequence of NEENA EN2393
SEQ ID NO: 17: Nucleotide sequence of NEENA EN2968
SEQ ID NO: 18: Nucleotide sequence of NEENA EN1391
SEQ ID NO: 19: Nucleotide sequence of NEENA EN4730
SEQ ID NO: 20: Nucleotide sequence of NEENA EN3681
SEQ ID NO: 21: Nucleotide sequence of NEENA EN5128
SEQ ID NO: 22: Nucleotide sequence of NEENA EN3638
SEQ ID NO: 23: Nucleotide sequence of NEENA EN2516
SEQ ID NO: 24: Nucleotide sequence of NEENA EN2161
SEQ ID NO: 25: Nucleotide sequence of NEENA EN2162
SEQ ID NO: 26: Nucleotide sequence of NEENA EN5096
SEQ ID NO: 27: Nucleotide sequence of NEENA EN5216
SEQ ID NO: 28: Nucleotide sequence of NEENA EN4689
SEQ ID NO: 29: Nucleotide sequence of NEENA EN2470
SEQ ID NO: 30: Nucleotide sequence of NEENA EN1451
SEQ ID NO: 31: Nucleotide sequence of NEENA EN1380
SEQ ID NO: 32: Nucleotide sequence of NEENA EN3504
SEQ ID NO: 33: Nucleotide sequence of NEENA EN3095
SEQ ID NO: 34: Nucleotide sequence of NEENA EN4474
SEQ ID NO: 35: Nucleotide sequence of NEENA EN1549
SEQ ID NO: 36: Nucleotide sequence of NEENA EN4245
SEQ ID NO: 37: Nucleotide sequence of NEENA EN1267
SEQ ID NO: 38: Nucleotide sequence of NEENA EN3758
SEQ ID NO: 39: Nucleotide sequence of NEENA EN4663
SEQ ID NO: 40: Nucleotide sequence of NEENA EN2195
SEQ ID NO: 41: Nucleotide sequence of NEENA ALMT1B
SEQ ID NO: 42: Nucleotide sequence of 1-kb promoter fragment of the 7A trehalose-6-phosphate phosphatase (T6PP) gene
SEQ ID NO: 43: Nucleotide sequence of 35S promoter nucleotides-208 to-65
SEQ ID NO: 44: Nucleotide sequence of ALMT1 3′
SEQ ID NO: 45: Nucleotide sequence of ALMT1 5′
SEQ ID NO: 46: Nucleotide sequence of lambda insulator fragment

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, CA, USA) using the Sanger technology (Sanger et al., 1977). Unless described otherwise, chemicals and reagents were obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases were from New England Biolabs (Ipswich, MA, USA) or Roche Diagnostics GmbH (Penzberg, Germany). Oligonucleotides were synthesized by Eurofins MWG Operon (Ebersberg, Germany). Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany. The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleo-monomers covalently coupled to each other by linkages (e.g., phosphodiesters) or substitute linkages.

### Example 1: Mapping of Functional Fragments of Selected Wheat Enhancer Sequences

An MPRA library (Melnikov et al. 2014, J. Vis. Exp. (90), e51719) was synthesized that contains for 6 previously identified wheat enhancers EN1390, EN3681, and EN5458 (described in WO 2021/48316 A1) and EN2516, EN3233 and EN5128 (described in WO 2021/110582 A1) the 144-nucleotide (nt) long sequence of the selected enhancer (herein referred to as “unmutated enhancer”) and each single-nucleotide mutant thereof, together with 2 positive control sequences (35S enhancer P35S-208 to-65 and ALMT1 3′, respectively SEQ ID NO 43 and 44) and 2 negative control sequences (ALMT1 5′ and lambda insulator fragment, respectively SEQ ID NO 45 and 46, WO 2021/48316 A1). Each test sequence was linked to 19 different 11-nt long barcodes. These sequences were cloned in a plasmid library as described in WO 2021/48316 A1, with the enhancer sequences upstream of the minimal 35S promoter and the barcodes downstream of a β-glucuronidase (GUS) gene that is under the control of the enhancer-minimal 35S promoter combination. A minimal promoter, as used herein, refers to promoter elements, particularly a TATA element, that are inactive or that have greatly reduced promoter activity in the absence of upstream activation. In the presence of a suitable transcription factor, the minimal promoter functions to permit transcription. The resulting plasmid library was transfected into wheat protoplasts and the frequency of the barcodes in the expressed RNA was compared to that in the plasmid library as a measure for the activity of the linked enhancer sequence (methods as described in WO 2021/48316 A1).

The ratio of the barcode's abundance in the RNA versus its' abundance in the plasmid library DNA is a measure for the expression enhancing activity of the test sequence that is linked to that specific barcode. As each test sequence is linked to 19 different barcodes, each test sequence has 19 RNA/DNA ratios. The median value of these RNA/DNA ratios was used as a measure for the enhancer activity of the tested sequence. As shown in Table 1, the 2 negative control sequences (ALMT1 5′ and lambda insulator fragment) had low RNA/DNA ratios (around 0.3), whereas the 35S enhancer had the highest RNA/DNA ratio and the RNA/DNA ratios of the wheat enhancer sequences were in-between these values. The second positive control sequence had a very low RNA/DNA ratio. This might be linked to the very low abundance of this sequence in the plasmid library DNA. EN3681 showed a similarly low abundance in the plasmid library and therefore this enhancer was not analyzed further.

TABLE 1
Abundance of the different WT test sequences
in the MPRA library compared to their abundance
in the expressed RNA in wheat protoplasts.

| Test sequence | DNA median | RNA median | RNA/DNA median |
|---|---|---|---|
| EN1390 | 28.81 | 37.68 | 1.32 |
| EN2516 | 22.52 | 14.36 | 0.72 |
| EN3233 | 8.96 | 17.53 | 1.60 |
| EN3681 | 3.85 | 4.28 | 1.19 |
| EN5128 | 34.00 | 22.72 | 0.75 |
| EN5458 | 29.48 | 28.71 | 1.03 |
| P35S -208 to-65 | 8.02 | 11.59 | 1.71 |
| Wheat ALMT1 3′ | 2.99 | 0.05 | 0.025 |
| Lambda insulator | 14.82 | 5.74 | 0.29 |
| Wheat ALMT1 5′ | 14.78 | 4.94 | 0.32 |

As was the case in the initial MPRA screening experiment (WO202148316A1, WO2021110582A1), EN3233 and EN1390 showed the highest RNA/DNA ratio of the unmutated enhancer sequences. In order to determine the impact of individual mutations on the enhancer's activity, the RNA/DNA ratio of the mutated sequences was compared to that of the unmutated reference sequence. This was done using a quantitative Enrichment ratio inference (ER) model from the Maphtic package (early version of MAVE-NN; Tareen et al. Genome Biology (2022) 23:98. https://doi.org/10.1186/s13059-022-02661-7).

For both EN1390 and EN3233, 2 smaller regions were identified that are putatively important for the enhancer activity in wheat protoplasts. For EN1390, these correspond to nt 65-70 and 81-97 (SEQ ID NO: 3) (FIG. 1), whereas for EN3233 these were mapped to nt 79-94 (SEQ ID NO: 9) and 106-126 (SEQ ID NO: 13) of the original sequence (FIG. 2). For each of these motifs, the results predict that the original wheat sequence is not optimal for enhancer activity and that an optimized sequence (see Table 2) would result in improved enhancer activity.

TABLE 2
Sequence motifs discovered by the MPRA mutational analysis of EN1390 and
EN3233 in wheat protoplasts. Both the wild-type sequence as it is present
in wheat, as well as the mutated sequence predicted to have the highest
activity are shown. Mutated nucleotides are shown in capitals.

| Enhancer | Position | Wheat sequence | Predicted optimal sequence (mut) |
|---|---|---|---|
| EN1390 | nt 65-70 | gataag | gataaT |
| EN1390 | nt 81-97 | atctgtcactgacgtgt | aCctgGGCAtgacgtgG |
| EN3233 | nt 79-94 | caggttcaacgaacgc | caggttcaacgaacgT |
| EN3233 | nt 106-126 | gtccaccagcgccagccgcct | AtcGaccaAcgccagccgcct |

For EN2516 and EN5128, the ER model did not identify any regions that are putatively important for the enhancer activity, whereas for EN5458 only a weak candidate region was identified for which no enhancer activity could be demonstrated in subsequent protoplast expression experiments.

### Example 2: Impact of EN1390 Fragments on Promoter Activity in Wheat Protoplasts

The MPRA experiment identified nt 65-70 and 81-97 of EN1390 as fragments that are putatively important for enhancer activity. To test whether these sequences indeed have enhancer activity, the following EN1390 sequences were cloned upstream of the minimal 35S promoter and the GUS coding sequence:
nt 61-76 (first motif including flanking sequences)
nt 81-97 (second motif)
nt 77-101 (second motif including flanking sequences)
nt 61-101 (first and second motif including flanking sequences).

TABLE 3
List of GUS expression vectors used to test the impact of various enhancer fragments on
promoter activity in wheat protoplasts. Fragments originate either from EN1390 or EN3233,
with the nucleotide position of the selected fragments in the original
enhancer sequence indicated. When positions run from high to low, the fragment
is tested in the reverse orientation. Nucleotides indicated
in capital letters deviate from the original wheat sequence.

| Construct | Promoter | Enhancer | Nt position | Tested sequence |
|---|---|---|---|---|
| pBay01697 | min35S | none | | |
| pBas03096 | min35S | EN1390 | 1-144 | tagacacgttgtgagctgccttgacttgggtaggcagatccagacctgaaa |
| | | | | cgtcaggctcatggataaggatcccggccatctgtcactgacgtgtcgtca |
| | | | | gtttgcccctagccttgtgggattgccatctccatctgccta |
| pBas04444 | min35S | EN1390 | 61-101 | catggataaggatcccggccatctgtcactgacgtgtcgtc |
| pBas04445 | min35S | EN1390 | 77-101 | ggccatctgtcactgacgtgtcgtc |
| pBas04446 | min35S | EN1390 | 81-97 | atctgtcactgacgtgt |
| pBas04447 | min35S | EN1390 | 61-76 | catggataaggatccc |
| pBas03112 | min35S | EN3233 | 1-144 | atgtatgcagccgtctcctgctgcttcactcaaggacgccacgccatccac |
| | | | | gcatatccggtggcggcaaacgccgtccaggttcaacgaacgccgttccc |
| | | | | cccggtccaccagcgccagccgcctccggccgggcccacccgc |
| pBas04439 | min35S | EN3233 | 74-132 | ccgtccaggttcaacgaacgccgttccccccggtccaccagcgccagccg |
| | | | | cctccggcc |
| pBas04440 | min35S | EN3233 | 74-100 | ccgtccaggttcaacgaacgccgttcc |
| pBas04441 | min35S | EN3233 | 102-132 | cccggtccaccagcgccagccgcctccggcc |
| pBas04442 | min35S | EN3233 | 79-94 | caggttcaacgaacgc |
| pBas04443 | min35S | EN3233 | 106-126 | gtccaccagcgccagccgcct |
| pBay02547 | T6PP | none | | |
| pBas03401 | T6PP | EN1390 | 1-144 | tagacacgttgtgagctgccttgacttgggtaggcagatccagacctgaaa |
| | | | | cgtcaggctcatggataaggatcccggccatctgtcactgacgtgtcgtca |
| | | | | gtttgcccctagccttgtgggattgccatctccatctgccta |
| pBas04669 | T6PP | EN1390 | 61-101 | catggataaggatcccggccatctgtcactgacgtgtcgtc |
| pBas04671 | T6PP | EN1390 | 61-101mut | catggataaTgatcccggccaCctgGGCAtgacgtgGcgtc |
| pBas03522 | T6PP | EN3233 | 1-144 | atgtatgcagccgtctcctgctgcttcactcaaggacgccacgccatccac |
| | | | | gcatatccggtggcggcaaacgccgtccaggttcaacgaacgccgttccc |
| | | | | cccggtccaccagcgccagccgcctccggccgggcccacccgc |
| pBas04756 | T6PP | EN3233 | 74-100 | ccgtccaggttcaacgaacgccgttcc |
| pBas04757 | T6PP | EN1390/3233 | 61-101/ | catggataaggatcccggccatctgtcactgacgtgtcgtcccgtccaggt |
| | | | 74-100 | tcaacgaacgccgttcc |
| pBas05140 | T6PP | EN3233/1390 | 74-100/ | ccgtccaggttcaacgaacgccgttcccatggataaggatcccggccatct |
| | | | 61-101 | gtcactgacgtgtcgtc |
| pBas05141 | T6PP | EN1390/3233 | 61-101/ | catggataaggatcccggccatctgtcactgacgtgtcgtcggaacggcgt |
| | | | 100-74 | tcgttgaacctggacgg |
| pBas05142 | T6PP | EN1390/3233 | 101-61/ | gacgacacgtcagtgacagatggccgggatccttatccatgccgtccagg |
| | | | 74-100 | ttcaacgaacgccgttcc |
| pBas05143 | T6PP | EN1390/3233 | 61-101mut/ | catggataaTgatcccggccaCctgGGCAtgacgtgGcgtcccgtcca |
| | | | 74-100 | ggttcaacgaacgccgttcc |

The resulting plasmids were introduced in wheat mesophyll protoplasts, protein was extracted, and GUS activities determined following an overnight incubation of the protoplasts. To correct for differences in introduction efficiency, GUS activities of transfected wheat protoplasts were divided by the luciferase activities from a co-introduced control vector having the firefly luciferase gene under control of the maize ubiquitin promoter (pKA63, WO202148316A1). Wheat protoplast preparation and PEG transfection of wheat protoplasts was performed according to Shang et al. (2014, Nature protocols 9 (10), 2395-2410).

The resulting data show that the 41-nt fragment containing both motifs (nt 61-101, SEQ ID NO: 5) has 66% of the enhancer activity of the complete 144-nt long EN1390 sequence (SEQ ID NO: 1) (FIG. 3A).

To test whether this 41-nt EN1390 fragment also increases the activity of other promoters this fragment (SEQ ID NO: 5) was inserted into a 1-kb promoter fragment of the wheat 7A trehalose-6-phosphate phosphatase (T6PP) gene (Liu and Zhang, 2018; SEQ ID NO: 42) at the same position as where the 144-nt long EN1390 was inserted before (WO202148316A1). In addition, a variant sequence that has the predicted optimal sequence for enhancer activity (61-101mut, Table 3, SEQ ID NO: 6) was inserted at the same position. Transfection of the resulting plasmids in wheat mesophyll protoplasts showed that the 41-nt wt fragment has about 50% of the enhancer activity of the complete 144-nt long EN1390 sequence, whereas optimization of the sequence of the 41-nt fragment increases the activity to almost 90% of the original EN1390 fragment (FIG. 3B). This thus identifies a sequence that is only 41-nt long and strongly increases the activity of a wheat promoter upon insertion within the promoter.

### Example 3: Impact of EN3233 Fragments on Promoter Activity in Wheat Protoplasts

The MPRA experiment identified nt 79-94 (SEQ ID NO: 9) and 106-126 (SEQ ID NO: 13) of EN3233 as fragments that are putatively important for enhancer activity, with the first fragment showing the highest contribution. To test whether these sequences have indeed enhancer activity, the following EN3233 sequences were cloned upstream of the minimal 35S promoter and the gus coding sequence:
nt 79-94 (first motif)
nt 74-100 (first motif including flanking sequences)
nt 106-126 (second motif)
nt 102-132 (second motif including flanking sequences)
nt 74-132 (first and second motif including flanking sequences).

Transfection of the resulting plasmids in wheat mesophyll protoplasts showed that the 27-nt fragment consisting of the first motif and some flanking nucleotides (nt 74-100, SEQ ID NO: 11) has more than 90% of the enhancer activity of the complete 144-nt long EN3233 sequence, whereas the contribution of the second motif to enhancer activity is small (FIG. 4). This thus identifies a sequence that is only 27-nt long and strongly increases promoter activity.

### Example 4: Combining EN1390 and EN3233 Enhancer Fragments Creates an Enhancer with Very Strong Activity in Wheat Protoplasts

Example 2 and 3 identified relatively small sequences that have strong enhancer activity in wheat protoplasts. Subsequently, a 68-nt long sequence, consisting of the 41-nt EN1390 fragment (SEQ ID NO: 5) followed by the 27-nt long EN3233 fragment (SEQ ID NO: 11), was cloned in the wheat T6PP promoter (SEQ ID NO: 42) and its effect on promoter activity in wheat protoplasts was compared with that of the individual fragments. This showed that the combined fragment had a much stronger enhancing effect than what would be expected based on the enhancing activity of each individual fragment, resulting in a 70- to 120-fold increased promoter activity (FIG. 5). Similarly high or even higher enhancing activities were observed when the two fragments were placed in a different configuration, such as a different order or, when one of the two fragments were placed in the reverse orientation, or when the optimized version of the EN1390 fragment (SEQ ID NO: 6) was used (FIG. 6). This thus delivers several 68-nt long enhancer sequences that strongly increase the activity of a wheat promoter to which the enhancer is functionally linked.

1. A method for enhancing expression derived from a plant promoter, comprising functionally linking to the promoter two or more heterologous nucleic acid expression enhancing nucleic acid (NEENA) molecules, wherein a first NEENA molecule and a second NEENA molecule share a nucleic acid sequence identity of at most 80%, said NEENA molecules comprising:
i) a nucleic acid having a sequence of any one of SEQ ID NOs: 1-41 or their complement, or
ii) a nucleic acid having at least 90% sequence identity to any one of SEQ ID NOs: 1-41 or their complement, or
iii) a nucleic acid hybridizing under stringent conditions to any one of SEQ ID NOs: 1-41 or their complement, or
iv) a functional fragment of 27 or more consecutive bases of a nucleic acid of i) to iii),
wherein the expression derived from said plant promoter is enhanced by 50% or more as compared to said plant promoter functionally linked to only said first or said second NEENA molecule.

2. A method for producing a plant or part thereof with enhanced expression of one or more nucleic acid molecules, as compared to a respective control plant or part thereof, comprising the steps of:
a) introducing into the plant or part thereof two or more heterologous NEENA molecules, wherein a first NEENA molecule and a second NEENA molecule share a nucleic acid sequence identity of at most 80%, said NEENA molecules comprising a nucleic acid molecule as defined in claim 1 i) to iv), and
b) functionally linking said two or more NEENA molecules to a promoter and to a nucleic acid molecule being under the control of said promoter, wherein said NEENA molecules are heterologous to said promoter,
wherein said control plant or part thereof comprises said promoter functionally linked only to said first or said second NEENA molecule.

3. The method according to claim 1, wherein said first NEENA or said second NEENA comprises:
i) a nucleic acid having a sequence of any one of SEQ ID NOs: 1-14 or their complement, or
ii) a nucleic acid having at least 90% sequence identity to any one of SEQ ID NOs: 1-14 or their complement, or
iii) a nucleic acid hybridizing under stringent conditions to any one of SEQ ID NOs: 1-14 or their complement, or
iv) a functional fragment of 27 or more consecutive bases of a nucleic acid of i) to iii).

4. The method according to claim 1, wherein said first NEENA comprises:
i) a nucleic acid having a sequence of any one of SEQ ID NOs: 1-6, or their complement, or
ii) a nucleic acid having at least 90% sequence identity to any one of SEQ ID NOs: 1-6 or their complement, or
iii) a nucleic acid hybridizing under stringent conditions to any one of SEQ ID NOs: 1-6 or their complement, or
iv) a functional fragment of 27 or more consecutive bases of a nucleic acid of i) to iii);
and wherein said second NEENA molecule comprises:
i) a nucleic acid having the sequence of any one of SEQ ID NOs: 7-14, or their complement, or
ii) a nucleic acid having at least 90% sequence identity to any one of SEQ ID NOs: 7-14 or their complement, or
iii) a nucleic acid hybridizing under stringent conditions to any one of SEQ ID NOs: 7-14 or their complement, or
iv) a functional fragment of 27 or more consecutive bases of a nucleic acid molecule of i) to iii).

5. The method according to claim 1, comprising the steps of
a) introducing said two or more NEENA molecules into a plant or part thereof, and
b) integrating said NEENA molecules into the genome of said plant or part thereof whereby said NEENA molecules are functionally linked to an endogenous promoter and to each other, said NEENA molecules being heterologous to said promoter and, optionally,
c) regenerating a plant or part thereof comprising said NEENA molecules from said plant or part thereof.

6. The method according to claim 5, wherein said NEENA molecules are integrated into the genome of a plant or part thereof by applying genome editing technologies.

7. The method according to claim 6, wherein the genome editing technology comprises the introduction of single or double strand breaks near to the position where a NEENA molecule is to be integrated into the genome using nucleic acid guided nucleases, TALEN, homing endonucleases or Zink finger proteins and the introduction of a DNA repair template comprising a NEENA molecule and at its 3′ and 5′ end sequences essentially identical or complementary to the sequences upstream and downstream of the single or double strand break facilitating recombination at the position of the single or double strand break.

8. The method according to claim 6, wherein the genome editing technology comprises introduction of point mutations in the genome of the plant or part thereof thereby introducing the sequence of a NEENA molecule in the plant genome.

9. The method according to claim 1 comprising the steps of
a) providing an expression construct comprising two or more NEENA molecules, said NEENA molecules comprising a nucleic acid as defined in claim 1 i) to iv) functionally linked to said promoter heterologous to said NEENA molecules and
b) integrating said expression construct into the genome of said plant or part thereof and, optionally,
c) regenerating a plant or part thereof comprising said one or more expression constructs from said transformed plant or part thereof.

10. The method according to claim 1, wherein said NEENA molecules are functionally linked to said promoter upstream or downstream of the transcriptional start site of a nucleic acid molecule the expression of which is under control of said promoter.

11. The method according to claim 1, wherein said NEENA molecules are functionally linked to said promoter within the 5′UTR or within an intron of the nucleic acid molecule the expression of which is under control of said promoter.

12. The method according to claim 1, wherein said promoter is a tissue specific, developmental specific or inducible promoter of a nucleic acid molecule the expression of which is under control of said promoter.

13. The method according to claim 1, wherein said NEENA molecules are present in one or more of the configurations selected from the group consisting of head-to-head, head-to-tail, tail-to-head, tail-to-tail, and combinations thereof.

14. The method according to claim 1, wherein said NEENA molecules are separated from each other by a spacer sequence.

15. The method according to claim 14, wherein said spacer sequence comprises 1 to 50 nucleotides.

16. The method according to claim 1, wherein
i) said first NEENA molecule comprises a nucleic acid with at least 90% sequence identity to nucleotide 74 to 100 of SEQ ID NOs: 7-8 or a nucleic acid that hybridizes under stringent conditions to nucleotide 74 to 100 of SEQ ID NO: 7-8, and
ii) said second NEENA molecule comprises a nucleic acid with at least 90% sequence identity to nucleotide 61 to 101 of SEQ ID NOs: 1-2 or a nucleic acid that hybridizes under stringent conditions to nucleotide 61 to 101 of SEQ ID NOs: 1-2.

17. The method according to claim 1, wherein said promoter is functionally linked to additional copies of said two or more NEENA molecules.

18. A recombinant expression construct comprising two or more NEENA molecules functionally linked to a promoter and one or more expressed nucleic acid molecules, wherein said NEENA molecules are heterologous to said promoter, wherein a first NEENA molecule and a second NEENA molecule share a nucleic acid sequence identity of at most 80%, and wherein said NEENA molecules comprise a nucleic acid as defined in claim 1 i) to iv).

19. A recombinant expression vector comprising one or more recombinant expression constructs according to claim 18.

20. A transgenic cell or transgenic plant or part thereof comprising a recombinant expression construct according to claim 18.

21. The transgenic cell, transgenic plant or part thereof according to claim 20, selected or derived from bacteria, fungi, yeasts or plants.

22. A transgenic cell culture, transgenic seed, parts or propagation material derived from a transgenic cell or plant or part thereof according to claim 20.

23. A method for producing foodstuffs, animal feeds, seeds, pharmaceuticals or fine chemicals comprising using a transgenic cell culture, transgenic seed, transgenic plant, parts or propagation material derived from a transgenic cell or plant according to claim 22.
